(19) United States
(12) Patent Application Publication
(10) Pub. No.: US 2017/0089997 A1
(43) Pub. Date: Mar. 30, 2017

(54) AQUEOUS CONTRAST AGENTS FOR DYNAMIC MRI AND MRA

(71) Applicant: BOSTON MEDICAL CENTER CORPORATION, Boston, MA (US)

(72) Inventors: Hernan JARA, Boston, MA (US); Asim MIAN, Boston, MA (US); Stephan ANDERSON, Boston, MA (US); Osamu SAKAI, Boston, MA (US); Alex NORBASH, Weston, MA (US); Jorge SOTO, Boston, MA (US)

(73) Assignee: BOSTON MEDICAL CENTER CORPORATION, Boston, MA (US)

(21) Appl. No.: 15/312,620

(22) PCT Filed: May 14, 2015
(86) PCT No.: PCT/US15/30703

§ 371 Date: Nov. 18, 2016

Related U.S. Application Data
(60) Provisional application No. 62/000,048, filed on May 19, 2014.

(51) Int. Cl. G01R 33/56; A61B 5/055; A61B 5/00; G01R 33/563

(52) U.S. Cl. CPC G01R 33/5601; G01R 33/5602; G01R 33/5635; G01R 33/56341; G01R 33/56366; A61B 5/055; A61B 5/0044; A61B 5/0042

(57) ABSTRACT

In a first aspect this invention provides methods comprising administering an aqueous contrast agent to the vascular system of a subject, and performing a magnetic resonance scan to detect the MR signal enhancement effects of the aqueous contrast agent. In embodiments the magnetic resonance scan comprises applying at least one pulse sequence selected from a PD-weighted pulse sequence, a T1-weighted pulse sequence, a T2-weighted pulse sequence, and a D-weighted pulse sequence. In embodiments the magnetic resonance scan comprises applying a T1-weighted pulse sequence. A system for performing perfusion MRI comprising an aqueous contrast solution and an injection apparatus configured to provide a maximum injection rate of the aqueous contrast solution to a subject vascular system of at least about 5 ml/s. is also provided.

This application claims priority to U.S. Provisional Application No. 62/000,048, filed May 19, 2014, which is hereby incorporated herein.

### INTRODUCTION

MRI contrast agents are a group of contrast media used to improve the visibility of soft tissues, organs and vasculature with magnetic resonance imaging (MRI). MRI offers a wide array of intrinsic tissue contrast mechanisms for aiding in the diagnosis of disease and these can be explored by simple manipulation of the imaging parameter values; a.k.a. pulse sequence parameters. Nonetheless, exogenous MRI contrast agents offer additional diagnostic information that is not easily obtainable with standard nonenhanced MRI techniques. In particular, gadolinium-based contrast agents (GBCA) are routinely used in the practice for diagnosing tissue pathology via contrast enhanced (CE) MRI, for assessing the vascular supply and the capillary bed integrity of biological tissue via magnetic resonance perfusion (MRP), and for providing high-quality depictions of the arterial vasculature via contrast enhanced magnetic resonance angiography (CE-MRA). The exceptional contrast-enhancing properties of GBCAs result from their powerful MR relaxation properties in terms of shortening the longitudinal magnetization recovery time (T1) as well as the spin-echo and gradient-echo transverse magnetization decay times (T2 and T2*). When injected into the blood stream, the local blood T1 can be shortened by ten-to-twenty fold, depending on dose and the level of blood oxygenation, and T2 (and T2*) can be shortened by approximately tenfold, depending on dose and the main magnetic field strength (BO). GBCAs are most commonly employed as T1 and T2* contrast agents using T1- and T2*-weighted pulse sequences, respectively.

Simply stated, GBCAs are very useful and potent MR contrast agents because they cause changes in the relaxation properties of blood and the perfused tissue in the direction of the fast relaxation limit of the biological spectrum (T1<200 ms and T2, T2*≦1 ms). This is accomplished with transitional heavy metal Gd ions (Gd3+), which are potent MR relaxers because of their large magnetic dipole moment. To be biologically compatible and therefore clinically useful, Gd3+ ions are tightly bound to chelating molecules in order to form chemically stable compounds that mitigate the natural toxicity of free metal ions.

While GBCAs have many useful properties, GBCAs also have clinical limitations. Perhaps most significantly, GBCAs are contraindicated in several patient groups including patients with renal insufficiency or on dialysis, and pregnant patients. Furthermore, special precaution is indicated in patients with asthma, a history of allergies, and prior iodinated contrast agent or GBCAs reactions. Data has also demonstrated that GBCAs may accumulate in several organ systems of the body and the possible detrimental effects of this are of concern. (See for example: Hao, D., et al. (2012). “MRI contrast agents: basic chemistry and safety.” Journal of Magnetic Resonance Imaging 36(5): 1060-1071). Use of GBCAs also adds significantly to the total cost of MRI procedures. This increases the economic burden of MRI and may discourage its use in some instances.

Deli, M., et al., “Saline as the Sole Contrast Agent for Successful MRI-Guided Epidural Injections,” Cardiovascular Intervent. Radiol., Vol. 36, pp. 748-55 (2013) describes a comparison of MRI-guided epidural injections using either gadolinium-enhanced saline solution or sterile saline solution for documentation of the epidural location of the needle tip. The authors analyzed MRI-guided epidural injections performed using gadolinium-enhanced saline solution or sterile saline solution as a contrast agent. The authors concluded that sterile saline is suitable as the sole contrast agent for successful and safe percutaneous MRI-guided epidural drug delivery. The authors interpreted their results as suggesting that saline could be a suitable contrast agent for other MRI-guided percutaneous drug delivery procedures, such as hip injections, shoulder injections, facet joint injections, sacroiliac joint injections, and temporomandibular joint injections. The authors did not suggest or even mention the use of saline as a vascular contrast agent.

Badawi, S. M., et al., “Magnetized Water and Saline as a Contrast Agent to Enhance MRI Images,” New York Science Journal, Vol. 5, No. 1 (2012) investigated the use of magnetized water and magnetized saline as contrast agents. Magnetized saline was prepared using a permanent magnetic funnel. The authors report that MRI of the brain following introduction of magnetized saline made “clear changes” visible compared to MRI in the absence of magnetized saline. It is also noteworthy that this article appears to emphasize the importance of pre-magnetizing the saline and that it is the “magnetized” feature that may make the saline act as a contrast agent for delayed static T2-weighted MRI.

A physical mechanism for such image contrast enhancement is not discussed by the authors. Furthermore, these investigators did not perform dynamic MRI scanning with the saline injection and instead two static MRI acquisitions were performed; one pre magnetized saline injection and a second scan one hour later.

Young, I. R., et al., “Pre-Polarized Saline: An In Vivo Feasibility Study of a Potential Contrast Agent,” NMR in Biomedicine, Vol. 12, pp. 381-86 (1999) experimented with pre-polarizing saline for use as a contrast agent. In their experiments the authors used a section of plastic pipe immersed in a bath of water doped with CuSO₄, with the time constant adjusted to roughly mimic that of muscle so as to model crudely the presence of a vessel in such tissue. (Young at page 385, col. 1.) The authors concluded that the feasibility of achieving enhancement from substantial pre-polarization of saline in vivo has been demonstrated. It is noteworthy that Young emphasizes that pre-polarization is required for use of saline as a contrast agent in their artificial system. For example, Young emphasized that once polarized, a method of rapid transfer of the saline from the pre-polarizing magnet to the imaging system is also necessary in order to avoid excessive signal loss through T1 decay, which would critically limit enhancement.

Application Publication US 2013/0142724 Al of Fu-Nien Wang describes a method of using intravenously injected deuterium oxide (D₂O) (i.e., “heavy water”) as an MRI contrast agent. Deuterium (2H) is a nonradioactive, naturally abundant (0.0156%), quadrupole interacting nuclide with only 0.01 the sensitivity of 1 H. According to the inventor, blood flow can be assessed by dynamic MRI detection and comparing images before and after the administration of D₂O. The inventor explained that the signal alternation resulting from use of heavy water in saline derives from two effects of the heavy water. First, the blood perfusion will replace the original H with D. The total amount of H in imaging voxels will be reduced by this replacement effect. The decreased density of H will reduce the signal intensities of H MRI. Second, a chemical exchange phenomenon will occur between H and D and slow the T1 and T2 relaxation of H. Due to similar physical and chemical properties of D and H, the introduction of D₂O into H₂O results in an isotopic H-D exchange and leads to a production of semi-heavy water HDO. It is noteworthy that this patent application publication emphasizes that it is the presence of D₂O in “heavy saline” that causes signal enhancement. This contrast enhancement effect of D₂O is not a dipolar relaxation enhancement effect and thus the effect is distinct from the mechanism of enhancement that occurs with GBCAs. It is also distinct for the contrast mechanism of the invention herein, which is based on injecting regular non-heavy-water (¹H₂O) thus leading to T1 (T2) elongation; the physico-physiological mechanism being hemodilution with MR active water.

Lin, W., et al., “Experimental Hypoxemic Hypoxia: Effects of Variation in Hematocrit on Magnetic Resonance T2*-Weighted Brain Images,” Journal of Cerebral Blood Flow and Metabolism, Vol. 18, pp. 1018-21 (1998) and Lin, W., et al., “Effects of Acute Normovolemic Hemodilution on T2*-Weighted Images of Rat Brain,” MRM, Vol. 40, pp. 857-64 (1998) induced acute normovolemic hemodilution (HD) in anesthetized rats to assess the effect of changes in hematocrit (Hct) on signal intensity in T2*-weighted magnetic resonance (MR) images. The Lin articles emphasize that changes in the oxygen saturation of blood have been used to induce signal intensity changes in T2*-weighted MR images. However, the authors were concerned that in certain situations reduction in Hct concentration could confound MR results obtained from methods that depend on changes in the oxygen saturation. For this reason the authors used an experimental system in which arterial blood was removed and replaced by a 5% solution of bovine serum albumin (BSA) in isotonic sodium chloride. The authors chose 5% albumin because that is approximately the albumin concentration in normal human plasma. Thus, under the experimental system used by Lin, Hct levels in blood were varied but protein levels in plasma were not. Lin also used T2*-weighted MR images exclusively. Lin does not mention or suggest the use of an aqueous contrast agent in a method comprising administering an aqueous contrast agent to the vascular system of a subject, and performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence. Lin also does not mention or suggest methods comprising performing a magnetic resonance scan comprises applying a T₁-weighted pulse sequence.

Quarles, C. C. and Gore, J. C, “Repeatable first pass DSC-MRI measurements using saline as a reverse-effect contrast agent,” ISMRM Proceedings, Voll 17, p. 724 (2009) (Quarles and Gore) conducted experiments in which saline was used as a reverse-effect contrast agent for dynamic susceptibility contrast MRI. Quarles and Gore only report detection of R2*-signal changes (i.e., the T₂* parameter) in their system, just as was the case in the Lin articles discussed in the preceding paragraph.

The T₂*-parameter is sensitive to field inhomogeneity. The Quarles and Gore, and Lin systems rely on use of saline to reduce inhomogeneity by dilution of blood. In essence the mechanism of the contrast enhancing properties of saline in those systems is mediated through blood dilution. Quarles and Gore, and Lin do not mention or suggest the use of an aqueous contrast agent in a method that utilizes the aqueous contrast agent to reduce dipole-dipole mediated relaxation, namely T₁, T₂ (to an extent), D, and PD. This is likely because the authors appreciated that blood dilution alone is not sufficient to enhance T₁-, T₂-(to an extent), D-, and PD-weighted pulse sequences. Quarles and Gore, and Lin do not suggest a method comprising administering an aqueous contrast agent to the vascular system of a subject, and performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence. Quarles and Gore also do not mention or suggest methods comprising performing a magnetic resonance scan comprises applying a T₁-weighted pulse sequence. Quarles and Gore, and Lin also neither discloses nor suggests that an aqueous contrast agent can enhance dipole-dipole mediated relaxation.

While the art showed that saline-induced contrast effects could be detected with MRI in an anatomical space and that this effect could be used, for example, to guide an epidural injection, the art has not identified aqueous contrast agent (ACA) formulations, or

MRI scanning and image processing methods for using such ACAs for dynamic MRI and dynamic magnetic resonance angiography or venography (MRA or MRV) utilizing a proton density (PD)-weighted pulse sequence, a T₁-weighted pulse sequence, a T₂-weighted pulse sequence, or a diffusion coefficient (D)-weighted pulse sequence. Because of the several drawbacks of GBCAs there is a need in the art for alternative contrast agents for use in dynamic MRI, MRA and MRV procedures. The inventions provided herein meet these and other needs.

### SUMMARY

Gadolinium-based contrast agents can have risks including nephrogenic systemic fibrosis, allergic reactions and limitation of use during pregnancy. Normal saline (NS) is a nontoxic sodium chloride water solution that can significantly increase the MR relaxation times of blood via transient hemodilution. The examples report the results of in-vivo tests in the head of the potential of NS as a safer, exogenous perfusion contrast agent. In a HIPAA compliant prospective study approved by the local IRB: twenty patients were scanned with T₁-weighted inversion recovery (IR) sequences. The IR pulse sequence was run during and after the NS injection for up to 5min: 100 ml of NS was power injected via antecubital veins at 3-4 ml/s. Images were processed to map maximum enhancement area-under-the-curve, time-to-peak, and mean-transit-time. These were used to identify the areas showing significant NS injection related signal and to generate enhancement time curves. NS injection-related enhancement effects were observed in all patients, particularly in vascular intra- and extra-cranial tissues. Relative signal change in cortical gray matter and periventricular white matter were observed in the 10-30% range and these enhancement effects lasted for several minutes post injection. A measurable perfusion effect of up to 30% change relative to baseline has been demonstrated in-vivo in the human brain using NS as a contrast agent. Without wishing to be bound by theory, the contrast mechanism is believed to be an alteration of the T₁ relaxation time resulting from transient hemodilution. This report of the use of NS for dynamic T₁-weighted perfusion MRI is novel and inventive.

Accordingly, this invention provides aqueous contrast agents for use in MRI procedures. This invention also provides methods and systems for performing magnetic resonance scans. These and other inventions are described herein.

In some embodiments the invention provides methods comprising administering an aqueous contrast agent to the vascular system of a subject, and performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence. In some embodiments of the methods the magnetic resonance scan comprises applying a T₁-weighted pulse sequence. In some embodiments of the methods the magnetic resonance scan further comprises applying at least one pulse sequence selected from a PD-weighted pulse sequence, a T₂-weighted pulse sequence, and a D-weighted pulse sequence. In some embodiments the methods further comprise comparing the MR signals at a first period with the MR signals at a second period, wherein the first and second periods are different and are selected from before administration of the aqueous contrast agent, during administration of the aqueous contrast agent, and after administration of the aqueous contrast agent. In some embodiments of the methods the presence of the aqueous contrast agent in the vascular system of the subject causes at least one of an increase in proton density (PD), an elongation of the longitudinal magnetization recovery time (T₁), an elongation of the transverse magnetization decay time (T₂), and an increase in the diffusion coefficient (D) in the dipolar relaxation signal from the subject. In some embodiments of the methods the presence of the aqueous contrast agent in the vascular system of the subject causes an elongation of the longitudinal magnetization recovery time (T₁). In some embodiments of the methods the aqueous contrast agent enhanced dipolar relaxation signal from the subject is enhanced by at least about 20%. In some embodiments of the methods the aqueous contrast agent enhanced dipolar relaxation signal from the subject is enhanced by at least about 30%.

In some embodiments the aqueous contrast agent is saline solution. In some embodiments the aqueous contrast agent is distilled water. In some embodiments the aqueous contrast agent does not comprise blood protein. In some embodiments the aqueous contrast agent does not comprise albumin.

In some embodiments the methods further comprise generating a contrast-enhanced magnetic resonance image of the subject. In some embodiments the contrast-enhanced magnetic resonance image of the subject is generated by a method comprising forming a map of at least one of maximum enhancement (maxENH), area under the curve (AUC), time to peak (TTP), and mean transit time (MTT).

In some embodiments of the methods the magnetic resonance scan is a quantitative magnetic resonance scan. In some embodiments of the methods the magnetic resonance scan is a functional magnetic resonance scan. In some embodiments of the methods the magnetic resonance scan is a dynamic magnetic resonance scan. In some embodiments of the methods the magnetic resonance scan is a magnetic resonance angiographic (MRA) scan.

In some embodiments the methods comprise performing at least one of perfusion imaging, arterial spin labeling, and diffusion imaging. In some embodiments the methods comprise performing a magnetic resonance angiogram or venogram of the subject.

In some embodiments the subject is a member of at least one group in which use of a gadolinium containing contrast agent is contraindicated.

In some embodiments the methods comprise the signal enhancement is not dependent on the presence of elevated deuterium in the contrast agent.

In some embodiments the methods further comprise recording the MR signal enhancement effects of the aqueous contrast agent.

In some embodiments the invention provides systems for performing perfusion MRI, comprising an aqueous contrast solution and an injection apparatus configured to provide a maximum injection rate of the aqueous contrast solution to a subject vascular system of at least about 5 ml/s.

In some embodiments the invention provides systems for performing perfusion MRI, comprising an aqueous contrast solution, an injection apparatus, and a controller comprising a programming system to allow programming of an injection protocol that specifies an injection rate of the aqueous contrast solution to a vascular system of a subject at a maximum rate of at least about 5 ml/s.

In some embodiments the systems further comprise computer usable media having a computer readable program code embodied therein, said computer readable program code specifying injection of a bolus of the aqueous contrast agent into the vascular system of a subject at a maximum rate of at least about 5 ml/s.

In some embodiments the systems further comprise a processor configured to generate a contrast-enhanced magnetic resonance image of the subject by a method comprising generating a map of at least one of maximum enhancement (maxENH), area under the curve (AUC), time to peak (TTP), and mean transit time (MTT).

In some embodiments the invention provides methods comprising administering an aqueous contrast agent to the vascular system of a subject, and performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence.

In some embodiments the invention provides methods comprising administering an aqueous contrast agent to the vascular system of a subject, performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence, and recording the MR signal enhancement effects of the aqueous contrast agent.

In some embodiments the invention provides methods comprising administering an aqueous contrast agent to the vascular system of a subject, and performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence, wherein the magnetic resonance scan comprises applying a T₁-weighted pulse sequence.

In some embodiments the invention provides methods comprising administering an aqueous contrast agent to the vascular system of a subject, and performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence, wherein the magnetic resonance scan comprises applying a T₁-weighted pulse sequence, and wherein the magnetic resonance scan further comprises applying at least one pulse sequence selected from a PD-weighted pulse sequence, a T₂-weighted pulse sequence, and a D-weighted pulse sequence.

In some embodiments the invention provides methods comprising administering an aqueous contrast agent to the vascular system of a subject, and performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence, and further comprising comparing the MR signals at a first period with the MR signals at a second period, wherein the first and second periods are different and are selected from before administration of the aqueous contrast agent, during administration of the aqueous contrast agent, and after administration of the aqueous contrast agent.

In some embodiments the invention provides methods comprising administering an aqueous contrast agent to the vascular system of a subject, and performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence, wherein the presence of the aqueous contrast agent in the vascular system of the subject causes at least one of an increase in proton density (PD), an elongation of the longitudinal magnetization recovery time (T₁), an elongation of the transverse magnetization decay time (T₂), and an increase in the diffusion coefficient (D) in the dipolar relaxation signal from the subject.

In some embodiments the invention provides methods comprising administering an aqueous contrast agent to the vascular system of a subject, and performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence, wherein the magnetic resonance scan comprises applying a T₁-weighted pulse sequence, wherein the presence of the aqueous contrast agent in the vascular system of the subject causes an elongation of the longitudinal magnetization recovery time (T₁).

In some embodiments the invention provides methods comprising administering an aqueous contrast agent to the vascular system of a subject, and performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence, wherein the aqueous contrast agent enhanced dipolar relaxation signal from the subject is enhanced by at least about 20%.

In some embodiments the invention provides methods comprising administering an aqueous contrast agent to the vascular system of a subject, and performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence, wherein the aqueous contrast agent enhanced dipolar relaxation signal from the subject is enhanced by at least about 30%.

In some embodiments the invention provides methods comprising administering an aqueous contrast agent to the vascular system of a subject, and performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence, wherein the aqueous contrast agent is saline solution.

In some embodiments the invention provides methods comprising administering an aqueous contrast agent to the vascular system of a subject, and performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence, wherein the aqueous contrast agent is distilled water.

In some embodiments the invention provides methods comprising administering an aqueous contrast agent to the vascular system of a subject, and performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence, wherein the aqueous contrast agent does not comprise blood protein.

In some embodiments the invention provides methods comprising administering an aqueous contrast agent to the vascular system of a subject, and performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence, wherein the aqueous contrast agent does not comprise albumin.

In some embodiments the invention provides methods comprising administering an aqueous contrast agent to the vascular system of a subject, and performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence, further comprising generating a contrast-enhanced magnetic resonance image of the subject.

In some embodiments the invention provides methods comprising administering an aqueous contrast agent to the vascular system of a subject, and performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence, further comprising generating a contrast-enhanced magnetic resonance image of the subject, wherein the contrast-enhanced magnetic resonance image of the subject is generated by a method comprising forming a map of at least one of maximum enhancement (maxENH), area under the curve (AUC), time to peak (TTP), and mean transit time (MTT).

In some embodiments the invention provides methods comprising administering an aqueous contrast agent to the vascular system of a subject, and performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence, wherein the magnetic resonance scan is a quantitative magnetic resonance scan.

In some embodiments the invention provides methods comprising administering an aqueous contrast agent to the vascular system of a subject, and performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence, wherein the magnetic resonance scan is a functional magnetic resonance scan.

In some embodiments the invention provides methods comprising administering an aqueous contrast agent to the vascular system of a subject, and performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence, wherein the magnetic resonance scan is a dynamic magnetic resonance scan.

In some embodiments the invention provides methods comprising administering an aqueous contrast agent to the vascular system of a subject, and performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence, wherein the magnetic resonance scan is a magnetic resonance angiographic (MRA) scan.

In some embodiments the invention provides methods comprising administering an aqueous contrast agent to the vascular system of a subject, and performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence, wherein the method comprises performing at least one of perfusion imaging, arterial spin labeling, and diffusion imaging.

In some embodiments the invention provides methods comprising administering an aqueous contrast agent to the vascular system of a subject, and performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence, wherein the method comprises performing a magnetic resonance angiogram or venogram of the subject.

In some embodiments the invention provides methods comprising administering an aqueous contrast agent to the vascular system of a subject, and performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence, wherein the subject is a member of at least one group in which use of a gadolinium containing contrast agent is contraindicated.

In some embodiments the invention provides methods comprising administering an aqueous contrast agent to the vascular system of a subject, and performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence, wherein the signal enhancement is not dependent on the presence of elevated deuterium in the contrast agent.

In some embodiments the invention provides systems for performing perfusion MRI, comprising an aqueous contrast solution and an injection apparatus configured to provide a maximum injection rate of the aqueous contrast solution to a subject vascular system of at least about 5 ml/s.

In some embodiments the invention provides systems for performing perfusion MRI, comprising an aqueous contrast solution, an injection apparatus, and a controller comprising a programming system to allow programming of an injection protocol that specifies an injection rate of the aqueous contrast solution to a vascular system of a subject at a maximum rate of at least about 5 ml/s.

In some embodiments the invention provides systems for performing perfusion MRI, comprising an aqueous contrast solution, an injection apparatus, and a controller comprising a programming system to allow programming of an injection protocol that specifies an injection rate of the aqueous contrast solution to a vascular system of a subject at a maximum rate of at least about 5 ml/s, wherein the programming system comprises a computer.

In some embodiments the invention provides systems for performing perfusion MRI, comprising an aqueous contrast solution, an injection apparatus, and a controller comprising a programming system to allow programming of an injection protocol that specifies an injection rate of the aqueous contrast solution to a vascular system of a subject at a maximum rate of at least about 5 ml/s, wherein the programming system comprises a computer.

In some embodiments the invention provides systems for performing perfusion MRI, comprising an aqueous contrast solution, an injection apparatus, and a controller comprising a programming system to allow programming of an injection protocol that specifies an injection rate of the aqueous contrast solution to a vascular system of a subject at a maximum rate of at least about 5 ml/s, and further comprising a processor configured to generate a contrast-enhanced magnetic resonance image of the subject by a method comprising generating a map of at least one of maximum enhancement (maxENH), area under the curve (AUC), time to peak (TTP), and mean transit time (MTT).

### BRIEF DESCRIPTION OF THE DRAWINGS

FIGS. 1A and 1B: Modeling Transient Hemodilution (THD). (A) A two-dimensional plot of blood T₁ as function of Hct and [Alb] was generated using Eq. 1 and it serves as the base surface over which the THD phenomenon can be envisioned. Before the NS injection, blood is at the physiologic point (1) and upon injection, it rapidly moves to point (2), which corresponds to a much lower Hct and [Alb] and consequently, has a much longer T₁. (B) The normalized concentration function shown was used in the computer simulations to modulate the Hct and Albumin concentration temporal profiles: in this simulation during the NS injection, blood is diluted down to the pure NS level with a transient very long T₁. After the NS injection, the Hct and Alb levels start recovering towards the physiologic baseline and this has been modeled herein with a simple exponential function, which does not include the possibility of NS recirculation.

FIG. 2: Dynamic IR-TSE Imaging Methods. The dynIR-TSE pulse sequence (top) starts with a large flip angle radiofrequency pulse (RF0˜180°) followed by an inversion time (TI) during which the longitudinal magnetizations of all tissues recover freely (bottom left graph). This time is also used to interrogate other slices for high scanning efficiency. At the inversion time TI, an excitation pulse)(RF1=90°) is applied; it converts the partially recovered longitudinal magnetizations into transverse magnetizations, which are then interrogated at multiple spin echo times according with turbo spin echo (TSE) acquisition principle. A typical modulus image (bottom center) leads to pixel values, which are always positive; hence, the signal curves (bottom right) decrease reaching a null point and increase subsequently. Note that the null point of each tissue Tₙᵤₗₗ(t)=ln(2) T₁(t) changes dynamically with THD in proportion to T₁.

FIG. 3: Hemodynamic Map Generation. The time series of each voxel (top) is processed using algorithms to generate maps of maximum enhancement (maxENH), area under the curve (AUC), time-to-peak (TTP), and mean-transit-time (MTT). Note the distinctive WM-to-GM contrast in the maxENH and AUC maps consistent with the higher perfusion fraction of GM relative to WM.

FIG. 4: Visualization of THD via Sequential Dynamic Partial-AUC Mapping. Single-slice time series of partial AUC maps shows the cumulative and incremental THD effects at twelve time points starting from pre-injection (upper left corner). Clearly, THD perfusion effects are more prominent in GM, choroid plexus, and some bone marrow regions, which is consistent with their higher microvascularity relative to WM and subcutaneous fat.

FIGS. 5A and 5B: Selected ROI-Based Time Series: High Spatial Resolution and Low Temporal Resolution. (A) Using a maxENH map at the level of the body of the lateral ventricles, ROIs were chosen for graphing the temporal evolution (Δτ=18.75s) of the percent signal difference (ΔS see Eq. 6) of the head of the caudate nucleus (ROI-1), a mixture of GM and WM (ROI-2), and pure WM (ROI-3). (B) Under these experimental conditions (TI=700 ms, 100 ml, 4 ml/s), the maximum GM signal level exceeds 10%, the GM and WM mixture has an intermediate signal (5%), and the minimum signal (˜-2.5%) is observed in pure WM.

FIGS. 6A and 6B: Selected ROI-Based Time Series: Low Spatial Resolution and High Temporal Resolution. (A) Using a maxENH map at the level of the body of the lateral ventricles, ROIs were chosen for graphing the temporal evolution of the percent signal difference (ΔS see Eq. 6) of the head of the caudate nucleus (ROI-1), pure WM (ROI-2), and cortical GM (ROI-3). (B) Under these experimental conditions (TI=700 ms, 100 ml, 3 ml/s), the maximum GM signal levels exceed 10%, and the minimum signal (˜2%) is observed in pure WM. Note that this patient was scanned at higher temporal resolution (Δτ=10.7 s) albeit at the expense of lower spatial resolution than the patient in FIG. 5, hence the maps are blurrier.

FIGS. 7A and 7B: Selected ROI-Based Time Series: Extracranial Tissues. (A) Using a maxENH map crossing the mid-cerebellum and the maxillary sinuses, three ROIs were selected to demonstrate selected THD signal temporal dependences of representative extracranial tissues: ROI-1 nasal mucosa with a very strong THD signal, ROI-2 much weaker signal from the lateral pterygoid muscle, and the signal of ROI-3 corresponding to subcutaneous fat is negligible at all times. (B) Notably, the THD signals of the nasal mucosa are also much stronger than brain tissue signals and last longer.

FIGS. 8A, 8B, and 8C: Linear THD Model. (A) and (B): graphs show that for GM and WM, the THD induced time changes of ΔT₁ and ΔS follow the same temporal pattern of the normalized function c(t), which modulates Hct and [Alb]. (C) ΔS and ΔT₁ are linearly related.

FIG. 9: Key Pulse Sequence Parameters. Selected imaging parameters of the four pulse sequences used in this work are grouped according to spatial resolution, image contrast weighting, and acquisition variables. * Note that because this was an exploratory study the dynlR-TSE parameters were varied slightly from patient to patient in an effort to optimize in real time the spatio-temporal resolution tradeoffs for best visualization of THD effects. As such, the parameters listed above represent the best case obtained herein whereby: a) highest spatial resolution is of paramount importance for minimizing GM-WM partial volume effects and b) choosing TI judiciously so that the time of signal measurement is close to the null point of the tissues of interest, most importantly GM and WM.

FIG. 10: Selected qMRI Parameters at 1.5T: Water, NS and Selected Tissues.

### DETAILED DESCRIPTION

Unless otherwise defined herein, scientific and technical terms used in connection with the present disclosure shall have the meanings that are commonly understood by those of ordinary skill in the art. Further, unless otherwise required by context, singular terms shall include the plural and plural terms shall include the singular. The materials, methods, and examples are illustrative only and not intended to be limiting.

The methods and techniques of the present disclosure are generally performed according to conventional clinical and preclinical MRI and MRA methods well known in the art and as described in various general and more specific references that are cited and discussed throughout the present specification unless otherwise indicated. See, e.g., Tofts, P. Quantitative MRI of the brain: measuring changes caused by disease. (John Wiley & Sons, 2005) and Sourbron, S. (2010). “Technical aspects of MR perfusion.” European Journal of Radiology 76(3): 304.

### A. Introduction

MRI offers a wide array of intrinsic contrast mechanisms for aiding in the diagnosis of disease and these can be explored by simple manipulation of the pulse sequence parameters (1, 2). Nonetheless, administration of exogenous contrast agents offers additional diagnostic information that is not easily obtainable with standard non-enhanced MRI techniques and the development of exogenous contrast agents has a rich history that parallels that of MRI itself (3). In particular, gadolinium (Gd) based contrast agents are routinely used for diagnosing tissue pathology via contrast enhanced MRI, for assessing the vascular supply and the capillary bed integrity of biological tissue via magnetic resonance perfusion, and for providing high-quality depictions of the arterial vasculature via contrast enhanced magnetic resonance angiography. The exceptional contrast-enhancing properties of Gd based contrast agents result from their powerful MR relaxation properties in terms of shortening the longitudinal magnetization recovery time (T₁) as well as the spin-echo and gradient-echo transverse magnetization decay times (T₂ and T₂*). When injected into the blood stream, the local blood T₁ can be shortened by ten-to-twenty fold, depending on Gd concentration, and T₂ (and T₂*) can be shortened by approximately tenfold, depending on Gd concentration and the main magnetic field strength (B₀). Simply stated, Gd based contrast agents are very useful and potent MR contrast agents because they cause changes in the relaxation properties of blood and the perfused tissue in the direction of the fast relaxation limit of the biological spectrum (T₁<200 ms and T₂, T₂*≦1 ms). This is accomplished with transitional heavy metal Gd ions (Gd³⁺), which are potent MR relaxers because of their large magnetic dipole moment. To be biologically compatible and therefore clinically useful, Gd³⁺ ions are tightly bound to chelating molecules in order to form chemically stable compounds that mitigate the Gd toxicity (4).

At the opposite end of the biological MR relaxation spectrum, or equivalently at the slow relaxation limit, is pure liquid water, which is the base solvent of all biological soft tissue other than fat and contains only light chemical elements (H₂O). The uniquely slow MR relaxation properties of water (5) result from the high rotational and translational mobility of water molecules (6-8), which in turn lead to very short correlation times (long T₁) as well as slow transverse magnetization decay (long T₂) by motional narrowing. As such, aqueous fluids have extreme and distinct MR properties in the biological spectrum: specifically, the cerebrospinal fluid (CSF) has the highest proton density, the highest diffusion coefficient, as well as the longest MR relaxation times within the human body.

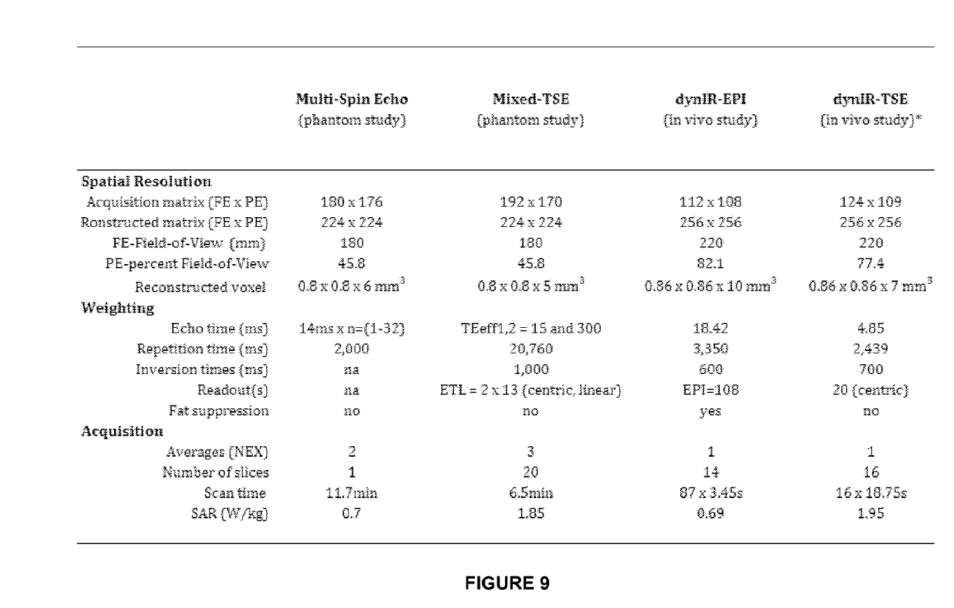

Normal saline (NS) is a clinically ubiquitous and nontoxic aqueous solution of sodium chloride (0.9% w/v) with quantitative MRI (qMRI) parameters not very different from those of pure water (9-11), but substantially different from blood, and very different from all normal soft tissue (FIG. 9). The inventors have discovered that NS injected intravenously (IV), alters the MR relaxation times and diffusion coefficient of blood via transient hemodilution (THD), which encompasses cell displacement (primarily erythrocytes, also leukocytes, and thrombocytes) as well as plasma-protein (primarily albumin) dilution. Such THD effects result in the following transient changes in blood: 1) elongation of the longitudinal magnetization relaxation time (T₁), 2) elongation of the transverse magnetization relaxation times (T₂ and T₂*), and 3) an increased diffusion coefficient (D). The THD of blood should causes qMRI changes of the perfused tissues. Consequently, NS acts as a diffusible contrast agent for T₁-, T₂-, and D-weighted MR imaging.

In this proof of concept investigation, the use of NS as a natural and safe intravascular T₁ contrast agent for perfusion weighted (PW) MRI of the human head is demonstrated. A quantitative justification for the experimental findings via computer simulations that model the ¹H₂O T₁ of blood and tissue during THD using a two-compartment blood relaxation model, blood (5, 11) and tissue (12-14) properties reported in the literature, and by solving the Bloch equation of the dynamic T₁-weighted pulse sequence is also provided.

Based in part on this demonstration, this invention provides methods comprising administering an aqueous contrast agent to the vascular system of a subject, and performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence. In some embodiments of the methods the magnetic resonance scan comprises applying a T₁-weighted pulse sequence. In some embodiments of the methods the magnetic resonance scan further comprises applying at least one pulse sequence selected from a PD-weighted pulse sequence, a T₂-weighted pulse sequence, and a D-weighted pulse sequence. In some embodiments the methods further comprise comparing the MR signals at a first period with the MR signals at a second period, wherein the first and second periods are different and are selected from before administration of the aqueous contrast agent, during administration of the aqueous contrast agent, and after administration of the aqueous contrast agent. In some embodiments of the methods the presence of the aqueous contrast agent in the vascular system of the subject causes at least one of an increase in proton density (PD), an elongation of the longitudinal magnetization recovery time (T₁), an elongation of the transverse magnetization decay time (T₂), and an increase in the diffusion coefficient (D) in the dipolar relaxation signal from the subject. In some embodiments of the methods the presence of the aqueous contrast agent in the vascular system of the subject causes an elongation of the longitudinal magnetization recovery time (T₁). In some embodiments of the methods the aqueous contrast agent enhanced dipolar relaxation signal from the subject is enhanced by at least about 20%. In some embodiments of the methods the aqueous contrast agent enhanced dipolar relaxation signal from the subject is enhanced by at least about 30%.

Additional embodiments of the methods and systems for implementation of the methods are also provided.

### B. Blood Under Normal Physiological Conditions

Blood is a complex “fluid-tissue” consisting of cells, primarily erythrocytes (red blood cells, RBC), leukocytes (white blood cells), and thrombocytes (platelets) that are transported in an aqueous solution of proteins and clotting factors known as plasma. Human plasma proteins include albumin (˜66%), alpha-1 (˜1%), alpha-2 (˜8%), beta (˜9%), and gamma (˜16%).

Despite the high diversity of its solute contents, the blood ¹H₂O longitudinal relaxation rate is well approximated by a simple two-compartment model with fast water exchange between the plasma compartment and the intracellular (IC) compartments.

R₁^{(blood)}=f₍ₚₗₐₛₘₐ₎r₁^{(plasma)}+f_{(IC)}R₁^{(IC)} Eq. A1

where the water fractions sum to unity.

Most researchers further simplify the two-compartment model by 1) limiting the intracellular space to that of the RBCs exclusively, and 2) by neglecting the relaxation effects of the less abundant plasma proteins, thus considering albumin only. Accordingly, in this reduced model, the relaxation rate of the longitudinal magnetization is a function of four primary variables specifically the hematocrit (Hct), the plasma albumin concentration ([Alb]), the oxygen saturation (Y), and the erythrocyte hemoglobin concentration ([Hb]) and given by:

R₁^{(blood)}(Hct,[Alb],Y,[Hb])=f₍ₚₗₐₛₘₐ₎(Hct)Rᵢ^{(plasma)}([Alb])+f_{(RBC)}(Hct)R₁^{(RBC)}(Y,[Hb]) Eq. A2

where the specific parameter dependencies of each term and factor are shown explicitly.

Furthermore, f₍ₚₗₐₛₘₐ₎ and f_{(RBC)} satisfy:

f₍ₚₗₐₛₘₐ₎(Hct)+f_{(RBC)}(Hct)=1 Eq. A3

Deoxyhemoglobin is a weak paramagnetic metalloprotein and R₁^{(RBC)}(Y, [Hb]) is approximately linearly related to the deoxygenation fraction (1-Y) via the mean corpuscular hemoglobin concentration ([Hb)) expressed in mmol Hb tetramer/L plasma in erythrocyte. Additional water relaxation effects result from the increased correlation times of water resulting from slow tumbling proteins, hence:

R₁^{(RBC)}(Y,[Hb])=R₁^{(NS)+r}_{1,Hb}[Hb]+r_{1,deoxyHb}[Hb](1−Y) Eq. A4

Similarly, the plasma relaxation rate can be written as:

R₁^{(plasma)}([Alb])=R₁^{(NS)+r}_{1,Alb}[Alb] Eq. A5

In the last two equations, the r_{1,x} denote the relaxivity of protein x dissolved in normal saline, which is the base solvent of blood. Hence, in this model, the relaxation mechanisms have been deconstructed down to the base intra-and extracellular biological solvent, which is normal saline; with R₁^{(Ns)}=0.222 Hz at physiological temperature.

Therefore, by combining the four preceding equations, we find:

R₁^{(blood)}(Hct,[Alb],Y,[Hb])=R₁^{(NS)}+(1−f_{(RBC)}(Hct))r_{1,Alb}[Alb]+f_{(RBC)}(Hct)(r_{1,Hb}+r_{1,deoxyHb}(1−Y))[Hb] Eq. A6

The fast exchange two-compartment model of blood is completed with the following additional equation:

$${f}_{( RBC )} ( Hct ) = \frac{0.70 Hct}{0.70 Hct + 0.95 ( 1 - Hct )} \qquad Eq . A7$$

which relates the intracellular water fraction to the hematocrit.

Formulating MR relaxation problems in terms of relaxation rates is mathematically advantageous because rates are additive quantities; however, relaxation times instead of relaxation rates are most often reported in the literature (Table 1), hence we recast the model (i.e. Eq. A6) as:

$${T}_{1}^{( blood )} ( Hct , [ Alb ] , Y , [ Hb ] ) == {[ \begin{matrix}\frac{1}{{T}_{1}^{( NS )}} + ( 1 - {f}_{( RBC )} ( Hct ) ) {r}_{1 , Alb} [ Alb ] + \\ {f}_{( RBC )} ( HCT ) ( {r}_{1 , Hb} + {r}_{1 , deoxyHb} ( 1 - Y ) ) [ Hb ]\end{matrix} ]}^{- 1} \qquad Eq . A8$$

This equation will be used to model the effects of transient hemodilution on T₁.

$${T}_{1}^{( blood )} ( Hct , [ Alb ] , Y , [ Hb ] ) {\to }^{THD} {T}_{1}^{( blood )} ( {t}_{dyn} ) {\to }^{Perfusion} {T}_{1}^{( tissue )} ( {t}_{dyn} ) \qquad Eq . A9$$

As a first approximation, we will assume that the intra-RBC THD effects can be neglected because the RBC content is near null during the thrust of the NS injection. Thus the main THD effects can be modelled as a temporal modulation of Hct and [Alb] with a common function of time, which we designate by c(t), accordingly:

$$\begin{matrix}{f}_{( RBC )} ( Hct ) {\to }^{THD} {f}_{( RBC )} ( {t}_{dyn} ) = \frac{0.70 Hct c ( {t}_{dyn} )}{0.70 Hct c ( {t}_{dyn} ) + 0.95 ( 1 - Hct c ( {t}_{dyn} ) )} and , & Eq . A10 \\ [ Alb ] {\to }^{THD} [ Alb ] ( {t}_{dyn} ) = [ Alb ] ( 0 ) c ( {t}_{dyn} ) . & Eq . A11\end{matrix}$$

### C. Transient Hemodilution in Blood

As an initial consequence of THD, the T₁ of blood becomes a function of time, which can be calculated using a streamlined two-compartment model with fast water exchange between the intra- and extracellular compartments (11); in such model (see Appendix):

$${T}_{1}^{( blood )} ( {t}_{dyn} ) == {[ \begin{matrix}\frac{1}{{T}_{1}^{( NS )}} + ( 1 - {f}_{( RBC )} ( {t}_{dyn} ) ) {r}_{1 , Alb} [ Alb ] ( {t}_{dyn} ) + \\ {f}_{( RBC )} ( {t}_{dyn} ) ( {r}_{1 , Hb} + {r}_{1 , deoxyHb} ( 1 - Y ) ) [ Hb ]\end{matrix} ]}^{- 1} \qquad Eq . 1$$

In this equation f_{(RBC)} is the red blood cell (RBC) water fraction, [Alb] is the concentration of plasma albumin, Y is the RBC oxygenation fraction, and each parameter r_{1,x} denotes the relaxivity of protein-x dissolved in normal saline, which is the base solvent of the intra- and extracellular compartments of blood. Hence, in this model, the relaxation mechanisms have been deconstructed down to the base intra-and extracellular biological solvent, which is normal saline; with T₁^{(NS)}=4,505 ms at 37° C.

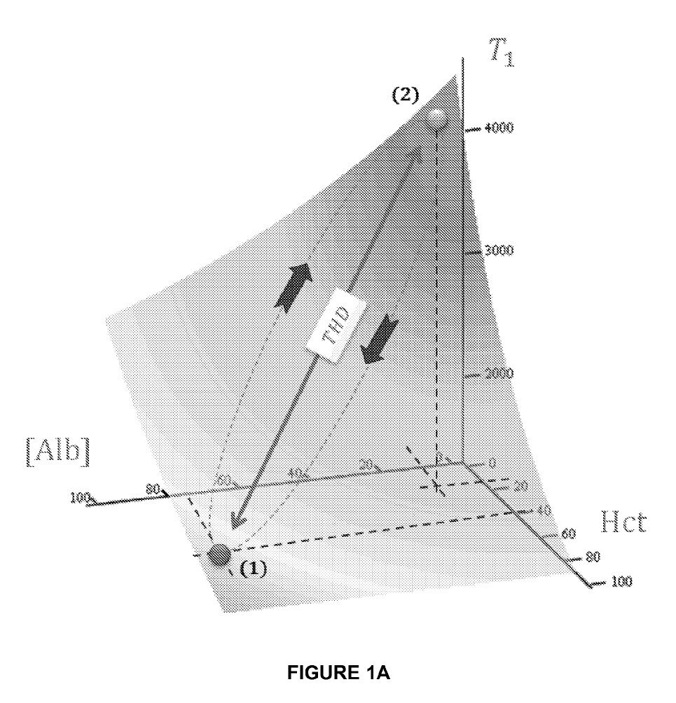

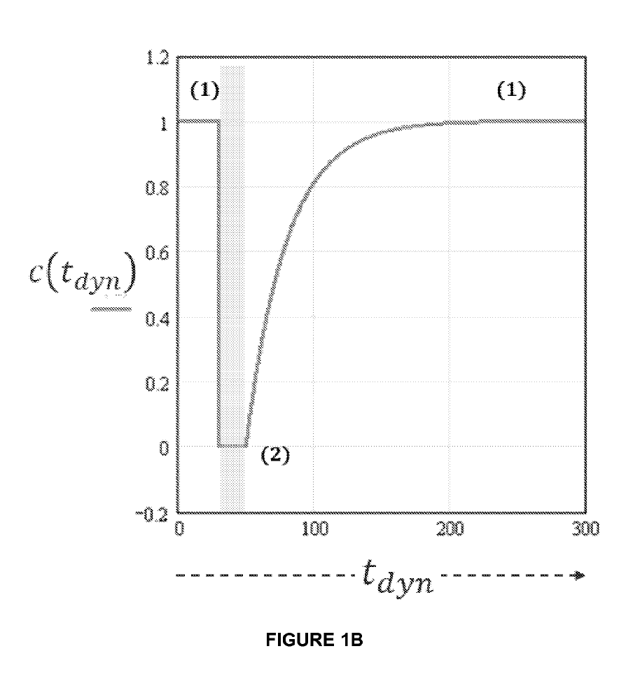

In addition, we have assumed in Eq. 1 that the intracellular THD relaxation effects due to dilution within the RBC cytoplasm can be neglected because the hematocrit becomes near null during the first pass of the NS injection. Thus the main THD effects can be modelled as a temporal modulation of Hct and [Alb] by a common normalized concentration function of time, which we designate by c(t), accordingly (also see FIG. 1):

$$\begin{matrix}{f}_{( RBC )} ( Hct ) {\to }^{THD} {f}_{( RBC )} ( {t}_{dyn} ) = \frac{0.70 Hct c ( {t}_{dyn} )}{0.70 Hct c ( {t}_{dyn} ) + 0.95 ( 1 - Hct c ( {t}_{dyn} ) )} and , & Eq . 2 a \\ [ Alb ] {\to }^{THD} [ Alb ] ( {t}_{dyn} ) = [ Alb ] ( 0 ) c ( {t}_{dyn} ) . & Eq . 2 b\end{matrix}$$

### D. Transient Hemodilution in Tissue

As the bolus of diluted blood reaches the capillary bed, the T₁ values of the perfused tissue also become dynamic variables, i.e.

$${T}_{1}^{( blood )} ( Hct , [ Alb ] , Y , [ Hb ] ) {\to }^{THD} {T}_{1}^{( blood )} ( {t}_{dyn} ) {\to }^{Perfusion} {T}_{1}^{( tissue )} ( {t}_{dyn} ) \qquad Eq . 3$$

and, to a first approximation neglecting NS migration into the extravascular space, these values can be calculated using the blood perfusion fraction fₜᵢₛₛᵤₑ and the following intravoxel partial-volume formula:

$${T}_{1}^{( tissue )} ( {t}_{dyn} ) = {( \frac{1 - {f}_{tissue}}{{T}_{1}^{( tissue )} ( 0 )} + \frac{{f}_{tissue}}{{T}_{1}^{( blood )} ( {t}_{dyn} )} )}^{- 1} \qquad Eq . 4$$

We hypothesize that such transient blood-tissue T₁ effects can be detected as pixel value temporal changes of a dynamic inversion recovery (IR) T₁-weighted pulse sequence that is run repetitively before, during, and after the NS injection. Such dynamic pixel values can be modelled via Bloch equation solution:

$$pv ( {T}_{( meas )} , {T}_{1} ( {t}_{dyn} ) ) ∝ {M}_{z}^{eq} \vert 1 - 2 exp ( - \frac{{T}_{( meas )}}{{T}_{1} ( {t}_{dyn} )} ) + 2 exp ( - \frac{( TR - \frac{TE}{2} )}{{T}_{1} ( {t}_{dyn} )} ) - exp ( - \frac{TR}{{T}_{1} ( {t}_{dyn} )} ) \vert \qquad Eq . 5$$

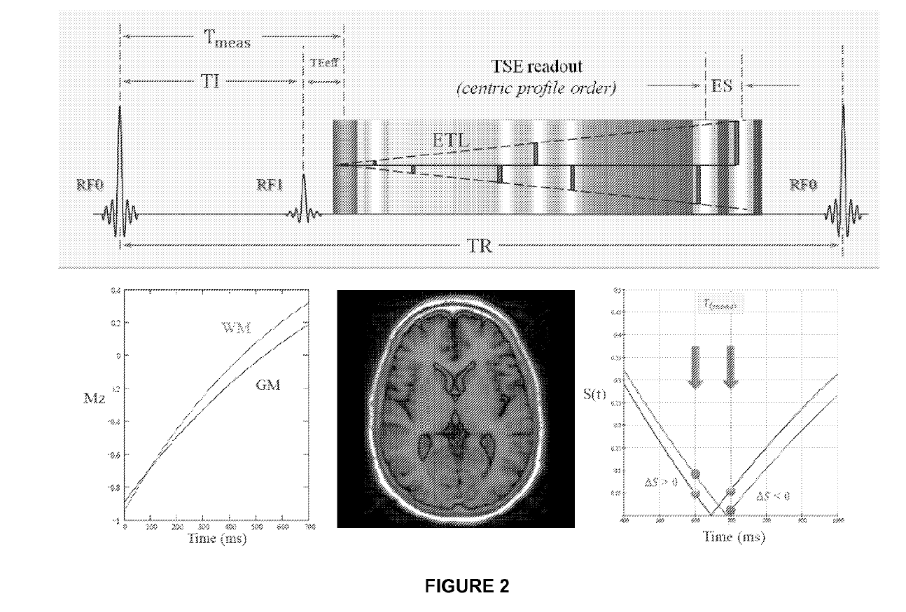

Here T₍ₘₑₐₛ₎=TI+TE_{eff} is the pulse sequence time of measurement at center of k-space (see FIG. 2) and the absolute value results from using modulus images. For this reason, the dynamic difference signals, specifically:

$$Δ S ( {T}_{( meas )} , {T}_{1} ( {t}_{dyn} ) ) = \frac{pv ( {T}_{( meas )} , {T}_{1} ( {t}_{dyn} ) ) - pv ( {T}_{( meas )} , {T}_{1} ( 0 ) )}{pv ( {T}_{( meas )} , {T}_{1} ( 0 ) )} \qquad Eq . 6$$

where T₁(0) is the pre-NS injection value. As shown graphically in FIG. 2 (bottom right), ΔS is positive if T₁(t_{dyn})>T₍ₘₑₐₛ₎/ln(2) and negative otherwise.

As the reduced-Hct bolus perfuses tissue, the local T₁ will consequently become elevated in proportion to its perfusion fraction and the tissue NS extraction rate. If the NS is injected at a sufficiently high rate, the diluted bolus could approximate the T₁ of pure saline at body temperature (Reference 9) (i.e. T₁˜4.5 s, see FIG. 1).

It is a further hypothesis that such transient T₁ lengthening phenomenon can be detected as time dependent pixel value change using a fast T₁-weighted pulse sequence run in dynamic mode during and after the NS injection. As demonstrated in the examples, this hypothesis is correct.

### E. Aqueous Contrast Agents

As used herein the term “aqueous contrast agent” means any aqueous solution that when administered intravascularly to a subject provides at least one of an increase in the mobile proton density (PD) that is detectable in an MRI scan of the subject, an elongation of the longitudinal magnetization recovery time (T₁) that is detectable in an MRI scan of the subject, an elongation of the transverse magnetization decay time (T₂ and/or T₂*) that is detectable in an MRI scan of the subject, and an increase in the diffusion coefficient (D) that is detectable in an MRI scan of the subject. In some embodiments “aqueous contrast agent” means any aqueous solution that when administered intravascularly to a subject provides an elongation of the longitudinal magnetization recovery time (T₁) that is detectable in an MRI scan of the subject. In general the at least one of an increase in proton density (PD), an elongation of the longitudinal magnetization recovery time (T₁), an elongation of the transverse magnetization decay time (T₂ and/or T_{2*}), and an increase in the diffusion coefficient (D) is by a factor of at least about 5%, at least about 10%, at least about 15%, at least about 20%, at least about 25%, or at least about 30% relative to what it would be in the absence of enhancement. There are many known methods that can be utilized to determine the degree of enhancement. For example any static or dynamic quantitative MRI technique of the proton density, the relaxation times (T1, T2, T2*), and the diffusion coefficient.

In some embodiments the aqueous contrast agent is a solution of from about 0.3% to about 1.5% w/v of NaCl in water. In some embodiments the aqueous contrast agent is a solution of from about 0.5% to about 1.3% w/v of NaCl in water. In some embodiments the aqueous contrast agent is a solution of from about 0.7% to about 1.1% w/v of NaCl in water. In some embodiments the aqueous contrast agent is a solution of about 0.9% w/v of NaCl in water (i.e., normal saline). In some embodiments the aqueous contrast agent is a solution of less than about 2% w/v of NaCl in water. In some embodiments the aqueous contrast agent is a solution of less than about 3% w/v of NaCl in water. In some embodiments the aqueous contrast agent is a solution of less than about 4% w/v of NaCl in water. In some embodiments the aqueous contrast agent is a solution of less than about 5% w/v of NaCl in water. In some embodiments the aqueous contrast agent is distilled water. In some embodiments the aqueous contrast agent is sterile pure water.

In some embodiments the aqueous contrast agent comprises at least one chemical component other than NaCl. In some embodiments the aqueous contrast agent comprises NaCl and at least one component other than NaCl. The at least one other component may be selected from at least one salt and at least one sugar, for example. In some embodiments the at least one other component is at least one GBCA. In some embodiments the at least one other component is present at a concentration of from about 0.1% to about 5%. In some embodiments the at least one other component is present at a concentration of from about 0.5% to about 5%. In some embodiments the at least one other component is present at a concentration of from about 1% to about 5%. In some embodiments the at least one other component is present at a concentration of from about 0.1% to about 0.5%. In some embodiments the at least one other component is present at a concentration of from about 0.1% to about 1%. In some embodiments the at least one other component is present at a concentration of less than about 5%. In some embodiments the at least one other component is present at a concentration of less than about 5%. In some embodiments the at least one other component is present at a concentration of less than about 4%. In some embodiments the at least one other component is present at a concentration of less than about 3%. In some embodiments the at least one other component is present at a concentration of less than about 2%. In some embodiments the at least one other component is present at a concentration of less than about 1%. In some embodiments the at least one other component is present at a concentration of less than about 0.5%. In some embodiments the at least one other component is present at a concentration of less than about 0.1%. In some embodiments the aqueous contrast agent comprises saline and does not comprise a GBCA.

In some embodiments the aqueous contrast agent is not pre-magnetized.

In some embodiments the aqueous contrast agent is not pre-polarized.

In some embodiments the aqueous contrast agent is saline solution.

In some embodiments the aqueous contrast agent is tap water.

In some embodiments the aqueous contrast agent is filtered tap water.

In some embodiments the aqueous contrast agent is distilled water.

In some embodiments the aqueous contrast agent does not comprise blood protein. In some embodiments the aqueous contrast agent does not comprise albumin.

In some embodiments the aqueous contrast agent does comprises no more than 1% blood protein. In some embodiments the aqueous contrast agent does comprises no more than 0.5% blood protein. In some embodiments the aqueous contrast agent does comprises no more than 0.1% blood protein.

In some embodiments the aqueous contrast agent does comprises no more than 1% albumin. In some embodiments the aqueous contrast agent does comprises no more than 0.5% albumin. In some embodiments the aqueous contrast agent does comprises no more than 0.1% albumin.

In some embodiments the aqueous contrast agent is used as a magnetic resonance angiography (MRA) or venography (MRV) contrast agent for visualizing all or some of the main body vessels of the body, e.g. the carotid arteries, the aorta, and the peripheral arteries of the arms and legs.

The aqueous contrast agent is administered into the vascular system of a subject to be scanned. For vascular administration, the ACA is injected intravenously or intra-arterially. The contrast agent may be administered at any rate desired for the particular application. In general, because of the lower viscosities of the aqueous contrast agents of the invention, ACAs may be administered at rates higher than those achievable with GBCAs. In some embodiments the aqueous contrast agent is administered at a rate of from about 1 ml/s to about 20 ml/s. In some embodiments the aqueous contrast agent is administered at a rate of from about 1 ml/s to about 5 ml/s. In some embodiments the aqueous contrast agent is administered at a rate of from about 5 ml/s to about 10 ml/s. In some embodiments the aqueous contrast agent is administered at a rate of from about 2 ml/s to about 4 ml/s. In some embodiments the aqueous contrast agent is administered at a rate of from about 2 ml/s to about 6 ml/s. In some embodiments the aqueous contrast agent is administered at a rate of from about 2 ml/s to about 8 ml/s. In some embodiments the aqueous contrast agent is administered at a rate of from about 2 ml/s to about 10 ml/s. In some embodiments the aqueous contrast agent is administered at a rate of from about 1 ml/s to about 10 ml/s. In some embodiments the aqueous contrast agent is administered at a rate of from about 10 ml/s to about 15 ml/s. In some embodiments the aqueous contrast agent is administered at a rate of from about 15 ml/s to about 20 ml/s. In some embodiments the aqueous contrast agent is administered at a rate of from about 10 ml/s to about 20 ml/s. In some embodiments the aqueous contrast agent is administered at a rate of from about 5 ml/s to about 15 ml/s.

In general a volume of from about 10 ml to about one liter or more of the aqueous contrast agent is administered as a single bolus. In some embodiments a volume of from about 20 ml to about 100 ml of the aqueous contrast agent is administered as a single bolus. In some embodiments a volume of from about 50 ml to about 100 ml of the aqueous contrast agent is administered as a single bolus. In some embodiments a volume of from about 100 ml to about 150 ml of the aqueous contrast agent is administered as a single bolus. In some embodiments a volume of from about 150 ml to about 200 ml of the aqueous contrast agent is administered as a single bolus. In some embodiments a volume of from about 200 ml to about 250 ml of the aqueous contrast agent is administered as a single bolus. In some embodiments a volume of from about 200 ml to about one liter of the aqueous contrast agent is administered as a single bolus. In some embodiments a volume of from about 200 ml to about 400 ml of the aqueous contrast agent is administered as a single bolus. In some embodiments a volume of from about 300 ml to about 500 ml of the aqueous contrast agent is administered as a single bolus. In some embodiments a volume of from about 400 ml to about 600 ml of the aqueous contrast agent is administered as a single bolus. In some embodiments a volume of from about 500 ml to about 700 ml of the aqueous contrast agent is administered as a single bolus. In some embodiments a volume of from about 600 ml to about 800 ml of the aqueous contrast agent is administered as a single bolus. In some embodiments a volume of from about 700 ml to about 900 ml of the aqueous contrast agent is administered as a single bolus. In some embodiments a volume of from about 800 ml to about one liter of the aqueous contrast agent is administered as a single bolus. In some embodiments a volume of from about 500 ml to about one liter of the aqueous contrast agent is administered as a single bolus.

The magnitude of aqueous contrast agent enhancement effect will generally be increased as the rate of injection is increased and/or the total volume injected is increased.

As used herein a “subject” is any animal comprising a vascular system suitable for administration of an aqueous contrast agent. The animal may be a human. The animal may be a non-human primate. The animal may be a farm or veterinary animal such as a horse, cow, sheep, pig, dog, or cat.

### F. MRI Pulse Sequences

Skilled artisans will appreciate that any MRI pulse sequence may be used to detect an intravascular contrast agent enhanced signal using the methods and systems of this invention. Non-limiting examples of pulse sequences that may be used to detect an enhanced T1 signal include inversion recovery (IR) with turbo spin echo (TSE), fast spin echo (FSE), gradient and spin echo (GraSE), and echoplanar imaging (EPI) readouts and short-TR pulse sequences (based on gradient echoes or spin echoes). Non-limiting examples of pulse sequences that may be used to detect an enhanced T2 weighted signal include medium (30-80 ms) and long (>80 ms) TE spin echo as well as FSE (TSE)). Non-limiting examples of pulse sequences that may be used to detect an enhanced T2* signal include medium and long TE gradient echo, GraSE, and gradient echo EPI. Non-limiting examples of pulse sequences that may be used to detect an enhanced D signal include variants of the pulsed field gradient diffusion-weighted (DW) technique: DW-SE, DW-TSE, and DW-Propeller. Non-limiting examples of pulse sequences that may be used to detect an enhanced PD signal include conventional (GE and SE) and hybrid (RARE, FSE, TSE, GraSE, and EPI) MRI pulse sequence with short TE and long TR. In some embodiments the pulse sequence used is designed to provide respiratory and/or cardiac motion compensation. In some embodiments the pulse sequence comprises an acceleration technique such as parallel imaging and/or simultaneous slice excitation.

### G. MRI Image Processing

Images generated from a dynamic MRI that utilizes an aqueous contrast agent in accordance with the invention may be processed using any suitable known procedure. Image processing may comprise subtracting the dynamic images of each slice from a reference image acquired at the beginning or the end of the procedure, performing pixel-by-pixel operations for generating perfusion related maps of at least one of maximum enhancement (maxENH), area under the curve (AUC), time to peak (TTP), and mean transit time (MTT).

### H. Types of Scans

The methods of this invention may in general be used to perform perfusion-enhanced magnetic resonance scan of any target tissue, such as without limitation a quantitative, functional, and/or dynamic magnetic resonance scan. Additional scans that may be performed include MRA and MRV. One non-limiting example is a magnetic resonance angiogram of a subject.

### I. Systems

Systems utilized in perfusion-enhanced MRI scans may comprise a contrast agent and an injection apparatus configured to provide an injection rate of the contrast agent into the vascular system of a subject or an animal. Because of the physical properties of GBCAs such agents are conventionally administered at rates of up to about 4-7 ml/s. Accordingly, systems that utilize GBCAs for performing perfusion-enhanced MRI scans are configured to inject the contrast agent at that rate. Because the lower viscosity aqueous contrast agents of this invention may be injected at faster rates and at up to a higher volume, the systems of this invention are typically configured to enable providing a higher contrast agent injection rate than can be achieved by prior art systems.

In some embodiments the system comprises a controller comprising a programming system to allow programming of an injection protocol that specifies an injection rate of the aqueous contrast solution to a vascular system of a subject. Again, because the aqueous contrast agents of this invention may be injected at faster rates and at up to a higher volume than prior art contrast agents, such systems typically comprise a programming system to allow programming of an injection protocol that specifies an injection rate of the aqueous contrast solution to a vascular system of a subject a rate that is higher than a rate appropriate for use with GBCAs.

Accordingly, this invention also provides a system for performing perfusion MRI, comprising an aqueous contrast solution and an injection apparatus configured to provide an injection rate of the aqueous contrast solution to a subject vascular system at a rate of up to at least about 20 ml/s.

This invention also provides a system for performing perfusion MRI, comprising an aqueous contrast solution, an injection apparatus, and a controller comprising a programming system to allow programming of an injection protocol that specifies an injection rate of the aqueous contrast solution to a vascular system of a subject at a rate of up to at least about 2 ml/s, up to at least about 3 ml/s, up to at least about 4 ml/s, up to at least about 5 ml/s, up to at least about 6 ml/s, up to at least about 8 ml/s, up to at least about 10 ml/s, up to at least about 12 ml/s, up to at least about 14 ml/s, up to at least about 16 ml/s, up to at least about 18 ml/s, or up to at least about 20 ml/s; or at a rate of from 2 ml/s to about 4 ml/s, at a rate of from 2 ml/s to about 6 ml/s, at a rate of from 2 ml/s to about 8 ml/s, at a rate of from 2 ml/s to about 10 ml/s, at a rate of from 4 ml/s to about 10 ml/s, at a rate of from 6 ml/s to about 10 ml/s, at a rate of from 10 ml/s to about 15 ml/s, or at a rate of from 15 ml/s to about 20 ml/s.

In some embodiments the programming system comprises a computer.

In some embodiments the system further comprises a computer usable media having a computer readable program code embodied therein, said computer readable program code specifying injection of a bolus of the aqueous contrast agent into the vascular system of a subject at a rate of up to at least about 2 ml/s, up to at least about 3 ml/s, up to at least about 4 ml/s, up to at least about 5 ml/s, up to at least about 6 ml/s, up to at least about 8 ml/s, up to at least about 10 ml/s, up to at least about 12 ml/s, up to at least about 14 ml/s, up to at least about 16 ml/s, up to at least about 18 ml/s, or up to at least about 20 ml/s; or at a rate of from 2 ml/s to about 4 ml/s, at a rate of from 2 ml/s to about 6 ml/s, at a rate of from 2 ml/s to about 8 ml/s, at a rate of from 2 ml/s to about 10 ml/s, at a rate of from 4 ml/s to about 10 ml/s, at a rate of from 6 ml/s to about 10 ml/s, at a rate of from 10 ml/s to about 15 ml/s, or at a rate of from 15 ml/s to about 20 ml/s.

In some embodiments the system further comprises a processor configured to generate a contrast-enhanced magnetic resonance image of the subject by a method comprising generating a map of at least one of maximum enhancement (maxENH), area under the curve (AUC), time to peak (TTP), and mean transit time (MTT).

The systems of the invention may be used to implement any of the methods of the invention.

### EXAMPLES

The following examples serve to more fully describe the manner of using the invention. These examples are presented for illustrative purposes and should not serve to limit the true scope of the invention.

### Materials and Methods

Subjects and Ethics

This HIPAA compliant prospective study was approved by the local Institutional Review Board, which authorized enrolling twenty outpatients under the conditions of injecting up to a maximum NS volume of 100 ml and with injection rates not exceeding 4 ml/s. All subjects signed a consent form. Inclusion criteria: 1) conscious outpatients older than 18 years that were scheduled for a non-emergent contrast enhanced MRI of the brain, and 2) patients who have the ability to consent in English. Patients with a history of congestive heart failure and pregnant patients were excluded. Twenty eligible patients, who were having an MRI of the brain for a variety of clinical indications, were successfully enrolled for this study.

Dynamic MR Imaging and Saline Injection

We used clinical 1.5T scanners (Philips Healthcare, Best, The Netherlands): 16 channel head and neck coil for signal reception and the quadrature body coil for radiofrequency transmission. An inversion recovery (IR) pulse sequence was run in synchronicity with the NS injections: 100 ml of NS were injected through an antecubital vein at a rate of 3-4 ml/s (3 ml/s employed in patients with a 22 Gauge needle; 4 ml/s in those with a 20 Gauge needle) using a power injector (Medrad Spectris Solaris, Bayer Healthcare, Warrendale, Pa.).

Phantom Experimentation

A sealed bag of NS was scanned for measuring the main qMRI parameters (PD, T₁, and T₂) at room temperature, using the mixed-TSE and a multi spin-echo pulse sequence (FIG. 9). In addition, a second phantom consisting of five 50 ml plastic vials embedded in agarose gel was used to test the temporal signal stability with the dynlR-TSE pulse sequence over a five-minute period. The vials contained iron doped agarose gels of different concentrations, and were surrounded by a bath of pure 3% agarose. The vials had the following iron concentrations: 0, 65, 130, 195, and 260 mg Fe/100 ml.

NS Signal Modelling and Image Processing

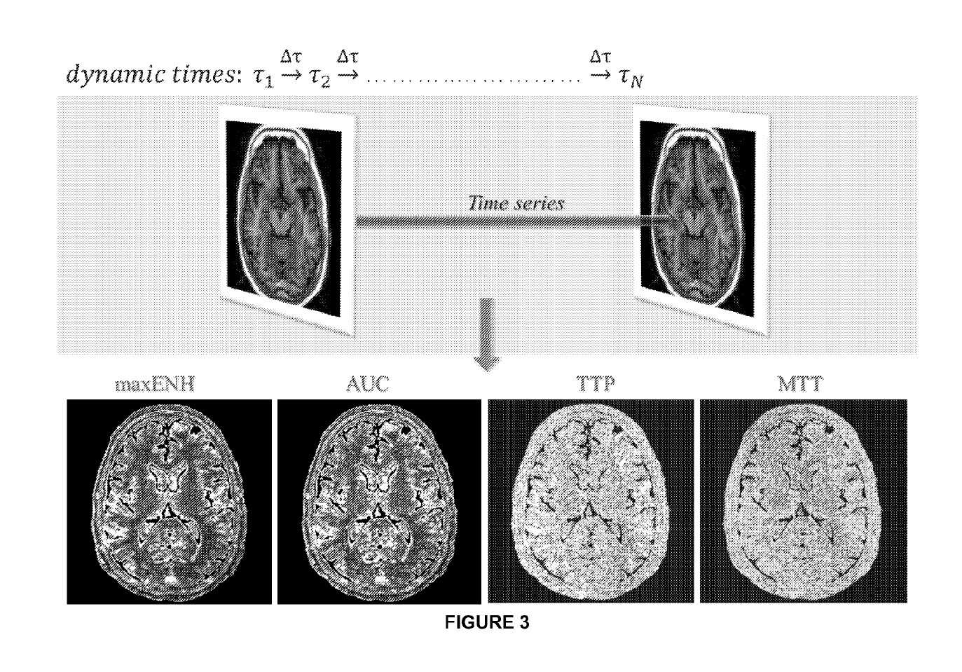

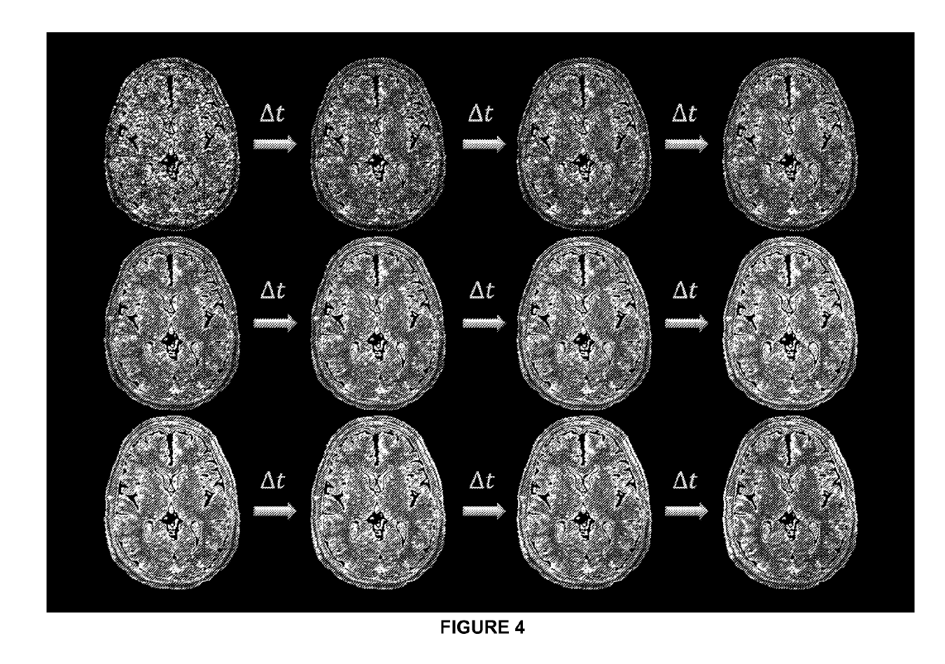

The volumetric time series in DICOM format were imported and realigned to the first dynamic set using the program Statistical Parametric Mapping version SPM8 (15) and converted into 16 bit binary images using ImageJ (http://imagej.nih.gov/ij/). The realigned time series were sorted by slice number and dynamic time of acquisition, followed by mapping the following dynamic parameters: maximum enhancement (maxENH), area under the curve (AUC), time-to-peak (TTP), and mean-transit-time (MTT) (FIG. 3). In house developed Mathcad algorithms (version 2001i, PTC, Needham, Mass.) were used for image sorting, THD modelling and perfusion parameter mapping. Additionally, a Mathcad algorithm was developed to generate dynamic partial-AUC time series, which provide a means for visualizing the incremental THD tissue effects as function of time (FIG. 4). The maxENH maps were used to identify the areas showing significant THD signal changes. Regions of interest (ROIs) were drawn in areas of high injection related effects as well as areas without these effects and graphed as a function of time.

### Example 1

### Phantom Experiments

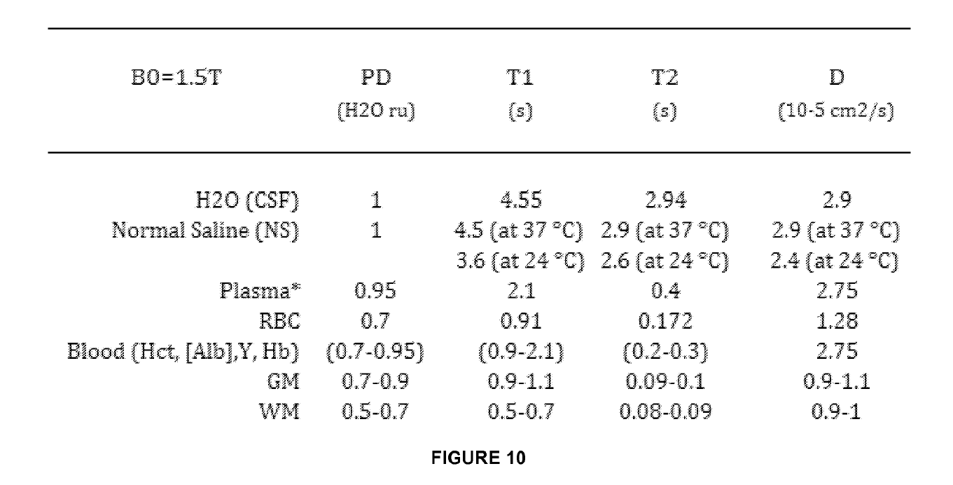

The measured qMRI parameters of NS are listed in FIG. 10 along with those of several head tissues and blood (5, 9, 11, 16-20): PD, T₁, and T₂ of NS are nearly identical to those of pure water and substantially different from the values of blood and brain tissues (21): gray matter (GM) and white matter (WM). The scan of the agarose phantom with the dynlR-TSE pulse sequence showed a very high temporal stability both with the large ROIs in several areas of the phantom as well as at the vial interfaces. Overall, temporal pixel intensity variations of less than 1% over the whole phantom during the five-minute scan were achieved with the dynlR-TSE pulse sequence.

### Example 2

### In Vivo Findings

The twenty enrolled subjects successfully completed the NS injection and the five-minute dynamic-MRI scan without experiencing any adverse effects or expressing any discomfort associated with the research portion of the MRI exam. This was a multivariable exploratory study geared at detecting a small perfusion related imaging effect in vivo and prospectively: as a result three different pulse sequences were tested and only twelve of the twenty subjects were scanned with the dynlR-TSE pulse sequence at 1.5T, which proved to be the most effective technique for detecting THD perfusion effects. The other eight subjects, which are not discussed further in this paper, were scanned with either an IR prepared T₁-weighted pulse sequence based on echo planar imaging (n=3), or with standard T₂-weighted TSE sequence (n=4) or at 3T (n=1).

### Example 3

### THD Signal Dynamics

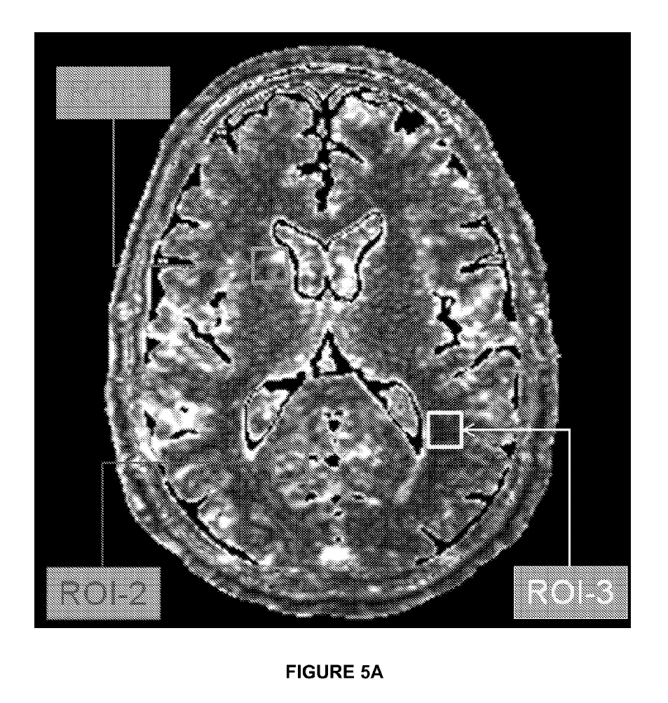

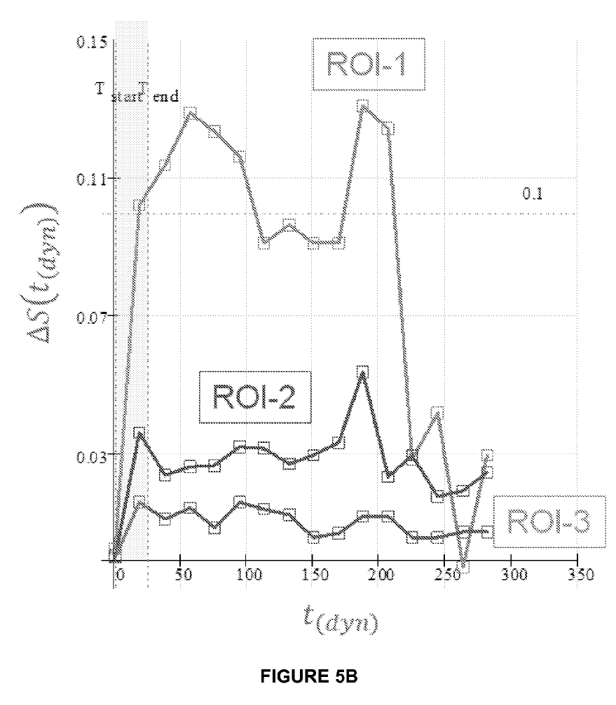

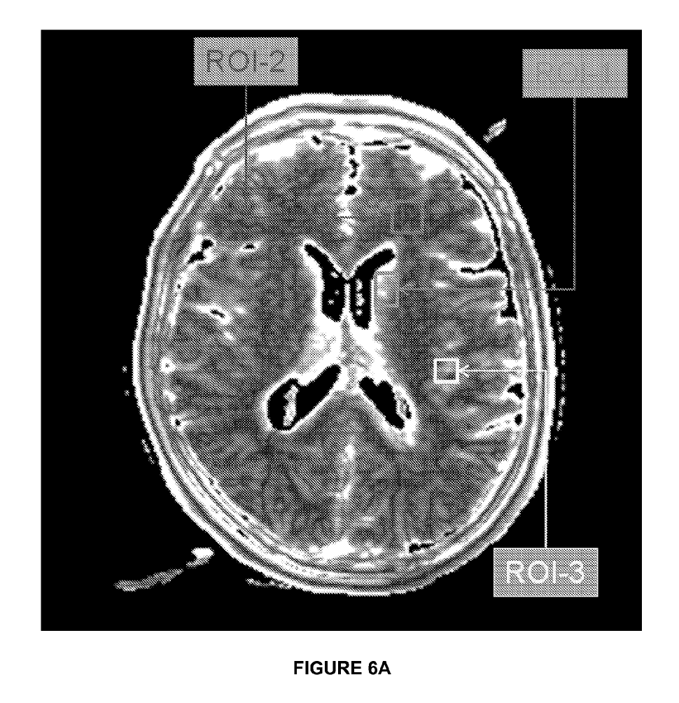

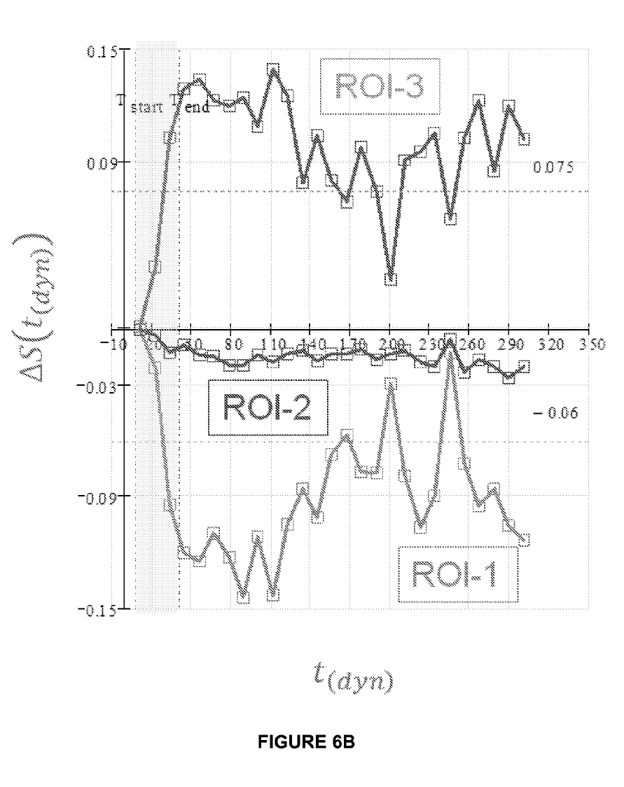

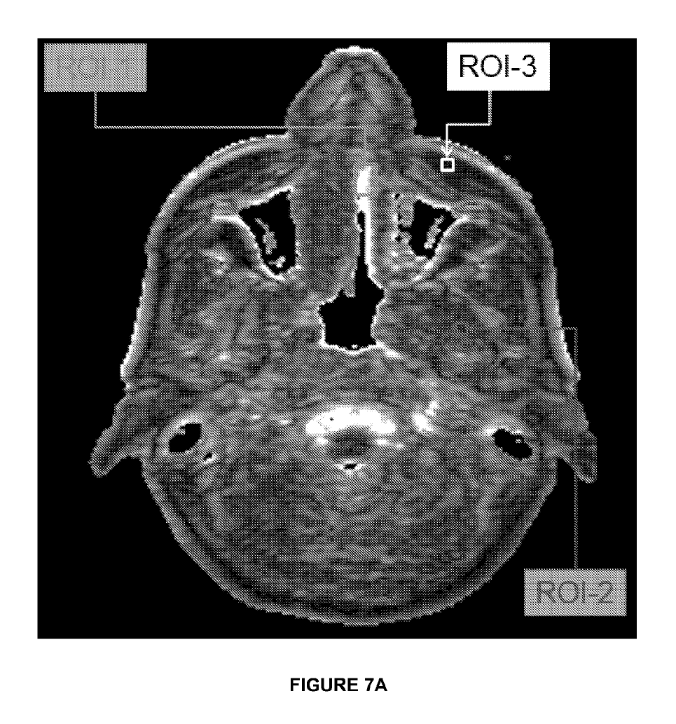

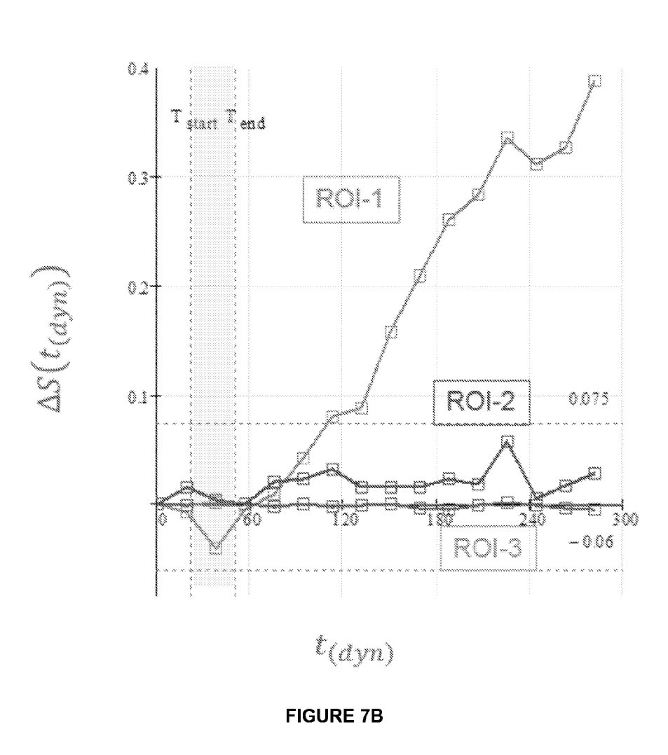

As shown in FIGS. 5 and 6, which correspond to two different patients, the brain tissue THD signals changed slowly time, extended over long times that far exceeded the injection time, and several (FIG. 7) even persisted past the five-minute observation time allowed in our protocol. With the injection parameters and imaging conditions used herein, the THD signal amplitudes were in the ranges of 10-15% and 2-5% for GM and WM respectively (FIGS. 5 and 6). Much stronger THD signals with amplitudes in the 20-30% range were observed in the nasal mucosa, which is consistent with the known higher vascularity of mucosal tissue relative to brain tissue. Other tissues that showed strong THD signals include the retina, choroid plexus, cranial bone marrow, and muscles. Pulsating arterial blood showed oscillatory signals that are likely modulated by inflow phenomena.

### Example 4

### Computer Simulations: Linear THD Model

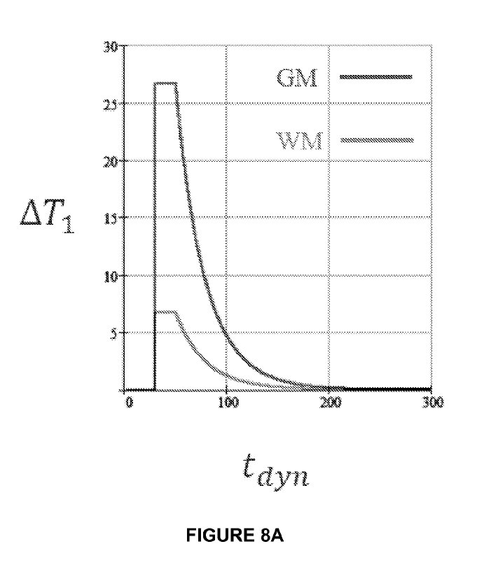

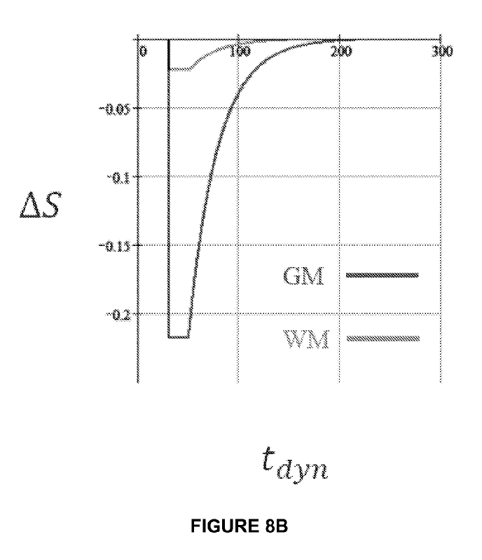

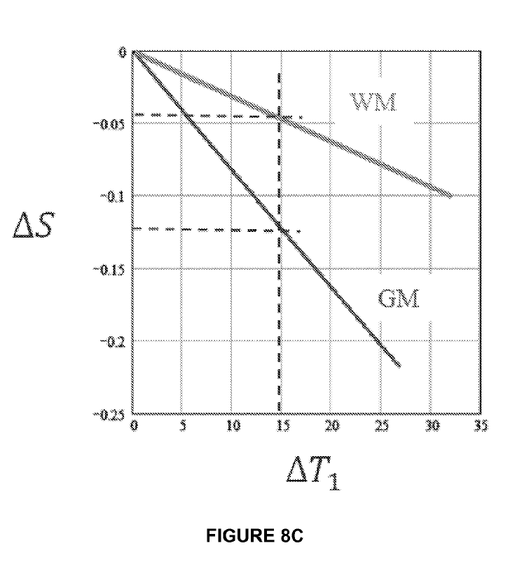

Using the tissue perfusion model of Eq. 4 through Eq. 6, the normalized tissue concentration function (FIG. 1B), and blood, GM (f=8%) and WM (f=4%) parameter values from the literature, we computed the THD induced relaxometric temporal changes ΔT₁ and the resulting signal changes ΔS as functions of time (FIGS. 8A and B): these graphs show that ΔT₁ and ΔS follow linearly the normalized concentration function. For each tissue ΔS varied linearly as a function of ΔT₁ with zero intercepts and slopes of: −122.71% signal ms⁻¹ and −333.66 signal ms⁻¹ for GM and WM respectively (FIG. 8C). Hence, this model predicts that for an absolute THD induced ΔT₁ of 15 ms, percent signal changes about 12% and 4% for GM and WM respectively: this is consistent with the ΔS values of FIGS. 5 and 6, and with those observed in other patients (not shown). In addition, these simulations were performed with different THD concentration functions, including a rectangular pulse and a gaussian pulse: in both cases, similar results were obtained. In particular, the linear dependence of ΔS as a function of ΔT₁ is independent of the temporal shape of c(t), thus providing support to the validity of the linear model as credible first approximation to the linear THD perfusion model.

### Discussion

Following administration of an NS injection, measurable hemodynamic MRI contrast enhancement effects secondary to THD have been demonstrated in-vivo in several intracranial and extracranial tissues of the human head. Measured THD signal levels ranged from a few percent for WM, to 8-15% for GM, to up to 35% for nasal mucosa and skin. As shown by ROI temporal analyses, the observed enhancement effects varied smoothly and slowly in time and lasted for several minutes post-injection. In some cases, contrast enhancement persisted past five minutes, which was the maximum scan time allowed per IRB protocol. We hypothesize that the physical mechanism of NS contrast enhancement is T₁ lengthening caused by THD. This physically intuitive hypothesis was further justified quantitatively by performing computer simulations based on: 1) using a two-compartment blood T₁ model, 2) averaging the relaxation rates of THD blood and perfused tissue with typical blood tissue fractions from the literature, and 3) by modelling the THD signals with the Bloch equation solution of the dynlR-TSE pulse sequence used. These simulations showed that the magnitude of the observed THD signals are in proportion to the tissue vascularity and in addition, correspond to T₁ differences of approximately 10 ms (FIG. 8) or ΔT₁/T₁˜1% for GM, clearly achievable with the rapid injection of 0.1 L in a total blood volume of 5 L.

Although the NS injection enhancement effects were not readily apparent in the source T₁-weighted images, the THD enhancement effects were clearly observable in the post-injection minus pre-injection subtraction images as well as in the hemodynamic parameter maps; in particular, in the maxENH and AUC maps in anatomic locations of the highly perfused tissues. Possible confounders to the NS signal enhancement effects could result from gross head motion, involuntary eye movements, and physiological motions (e.g. pulsations and intravascular inflow artifacts). Several confirmatory observations lead us to establish the veracity of the reported experimental observations and to confirm that these are genuine NS injection related enhancement effects. Phantom experiments attest as to the temporal stability of the scanning hardware and pulse sequence. For the nine patient scans that had minimal gross head motion, as confirmed by scrolling rapidly through the source images in repetitive cine mode, the following general observations are made. First, the enhancement signals followed the NS injection: to prove the point, in one case the injection time was purposely delayed by 2.5 min and the enhancement signals shifted in time accordingly. Second, the magnitudes of the enhancement effects correlated hierarchically with the known levels of tissue vascularity. Third, some tissues such as subcutaneous fat, the brain stem showed negligible enhancement signal throughout the 5 min scan.

Four fundamentally different perfusion MRI techniques have been developed in the past twenty-five years and these have been studied intensively (22-26) in the quest for accurate and reliable tissue perfusion assessment. Current perfusion MRI techniques include dynamic susceptibility weighted contrast (DSC), dynamic contrast enhancement (DCE), ASL, and intravoxel incoherent motion (IVIM). Of these, DSC and DCE are performed after the intravenous injection of a Gd-chelate contrast agent, which are probably the most potent, useful, and used T₁ (and T₂*) contrast agents available today (27, 28). However, these cannot be administered to patients with impaired renal function, eGFR of less than 30 ml/minute or allergy to Gd. In addition, some Gd-chelate contrast agents can have risks including nephrogenic systemic fibrosis, allergic reactions and are of limited use during pregnancy (28-32). These potential limitations motivate the search for other injectable contrast agents that could substitute Gd-based contrast agents in vulnerable populations. ASL and IVIM are very safe because they do not use injectable materials but instead rely on ingenious perfusion-weighted MRI pulse sequences.

Except for IVIM, all current perfusion MRI techniques use a tracer, which can be exogenous or endogenous, and these can be further classified as diffusible or nondiffusible based on their ability to permeate through the brain-blood-barrier. Gd-chelated compounds are considered nondiffusible, whereas arterial spin labeling (ASL) that labels the native protons in blood water is considered diffusible. Perfusion weighted MRI using THD via NS injections would join ASL in the diffusible tracer category of MRI perfusion weighted techniques.

The MRI perfusion field has not yet identified a single well-defined technique either that has no medical contraindications or that does not pose technical challenges:

including limited SNR in the case of ASL and high motion artifact vulnerability in the case of IVIM. To the best of our knowledge, the study herein is the first report of a use of NS as a dynamic intravascular MRI contrast agent in humans and it is therefore too early to attempt a meaningful comparison between the THD and existing perfusion techniques. Quarles and Gore reported NS use as intravascular T₂* contrast agent in rats (33). A non-intravascular MRI application of NS was reported in the context of MRI-guided epidural injections (34).

In conclusion, a measurable perfusion effect has been demonstrated in-vivo in the human brain using normal saline as an injectable contrast agent. The contrast mechanism is hypothesized to be an elongation of the T₁ relaxation time resulting from THD. The described methodology could translate into safely extending the use of exogenous contrast agents in MR perfusion by decreasing the contraindications that patients encounter, reducing costs, and extending the use of MRI for improving patient care.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### REFERENCES

1. Haacke E, Thompson M, Brown R. Magnetic resonance imaging: physical principles and sequence design: Wiley New York, 1999.
2. Tofts P. Quantitative MRI of the brain: measuring changes caused by disease: John Wiley & Sons, 2005.
3. Edelman R R. The history of MR imaging as seen through the pages of Radiology. Radiology. 2014;273(2S):S181-S200.
4. Hao D, Ai T, Goerner F, Hu X, Runge V M, Tweedle M. MRI contrast agents: basic chemistry and safety. Journal of Magnetic Resonance Imaging. 2012;36(5):1060-71.
5. Brooks R, Di Chiro G. Magnetic resonance imaging of stationary blood: a review. Medical Physics. 1987;14(6):903-13.
6. Bloembergen N, Purcell E, Pound R. Relaxation effects in nuclear magnetic resonance absorption. Physical Review, vol 73. 1948(7):679-712.
7. Torrey H. Nuclear spin relaxation by translational diffusion. Physical Review, vol 92. 1953(4):962-9.
8. Solomon I. Relaxation processes in a system of two spins. Physical Review. 1955;99(2):559-65.
9. d'Othée B J, Rachmuth G, Munasinghe J, Lang E V. The effect of hyperoxygenation on T1 relaxation time in vitro. Academic radiology. 2003;10(8):854-60.
10. Zaharchuk G, Martin A, Rosenthal G, Manley G, Dillon W. Measurement of cerebrospinal fluid oxygen partial pressure in humans using MRI. Magnetic Resonance in Medicine. 2005;54(1):113-21.
11. Grgac K, van Zijl P C M, Qin Q. Hematocrit and oxygenation dependence of blood 1H2O T1 at 7 tesla. Magnetic Resonance in Medicine. 2013;70(4):1153-9.
12. Wirestam R, Brockstedt S, Lindgren A, et al. The perfusion fraction in volunteers and in patients with ischaemic stroke. Acta Radiologica. 1997;38(6):961-4.
13. Wirestam R, Borg M, Brockstedt S, Lindgren A, Holtås S, Ståhlberg F. Perfusion-related parameters in intravoxel incoherent motion MR imaging compared with CBV and CBF measured by dynamic susceptibility-contrast MR technique. Acta Radiologica. 2001;42(2):123-8.
14. Federau C, O'Brien K, Birbaumer A, Meuli R, Hagmann P, Maeder P. Functional mapping of the human visual cortex with intravoxel incoherent motion MRI. PLoS One. 2015;10(2):e0117706.
15. Penny W D, Friston K J, Ashburner J T, Kiebel S J, Nichols T E. Statistical parametric mapping: the analysis of functional brain images: the analysis of functional brain images: Academic Press, 2011.
16. Barth M, Moser E. Proton NMR relaxation times of human blood samples at 1.5 T and implications for functional MRI. Cellular and molecular biology (Noisy-le-Grand, France). 1997;43(5):783-91.
17. Stanisz G J, Li J G, Wright G A, Henkelman R M. Water dynamics in human blood via combined measurements of T2 relaxation and diffusion in the presence of gadolinium. Magnetic Resonance in Medicine. 1998;39(2):223-33.
18. Spees W, Yablonskiy D, Oswood M, Ackerman J. Water proton MR properties of human blood at 1.5 Tesla: Magnetic susceptibility, T1, T2, T*2, and non-Lorentzian signal behavior. Magnetic Resonance in Medicine. 2001;45(4):533-42.
19. Silvennoinen M J, Clingman C S, Golay X, Kauppinen R A, van Zijl P C M. Comparison of the dependence of blood R2 and R2* on oxygen saturation at 1.5 and 4.7 Tesla. Magnetic Resonance in Medicine. 2003;49(1):47-60.
20. Silvennoinen M J, Kettunen M I, Kauppinen R A. Effects of hematocrit and oxygen saturation level on blood spin-lattice relaxation. Magnetic Resonance in Medicine. 2003;49(3):568-71.
21. Saito N, Sakai O, Ozonoff A, Jara H. Relaxo-volumetric multispectral quantitative magnetic resonance imaging of the brain over the human lifespan: global and regional aging patterns. Magn Reson Imaging. 2009;27(7):895-906.
22. Wintermark M, Sesay M, Barbier E, et al. Comparative overview of brain perfusion imaging techniques. Stroke. 2005;36(9):e83.
23. Sourbron S. Technical aspects of MR perfusion. European Journal of Radiology. 2010;76(3):304.
24. Detre J A, Rao H, Wang D J J, Chen Y F, Wang Z. Applications of arterial spin labeled MRI in the brain. Journal of Magnetic Resonance Imaging. 2012;35(5):1026-37.
25. McGehee B E, Pollock J M, Maldjian J A. Brain perfusion imaging: How does it work and what should I use? Journal of Magnetic Resonance Imaging. 2012;36(6):1257-72.
26. Günther M. Perfusion imaging. Journal of Magnetic Resonance Imaging. 2014;40(2):269-79.
27. Lin S-P, Brown J J. MR contrast agents: physical and pharmacologic basics. Journal of Magnetic Resonance Imaging. 2007;25(5):884-99.
28. Natalin R A, Prince M R, Grossman M E, Silvers D, Landman J. Contemporary applications and limitations of magnetic resonance imaging contrast materials. The Journal of Urology. 2010;183(1):27-33.
29. Ersoy H, Rybicki F J. Biochemical safety profiles of gadolinium-based extracellular contrast agents and nephrogenic systemic fibrosis. Journal of Magnetic Resonance Imaging. 2007;26(5):1190-7.
30. Wertman R, Altun E, Martin D R, et al. Risk of nephrogenic systemic fibrosis: evaluation of gadolinium chelate contrast agents at four american universities. Radiology. 2008;248(3):799-806.
31. Martin D R, Krishnamoorthy S K, Kalb B, et al. Decreased incidence of NSF in patients on dialysis after changing gadolinium contrast-enhanced MRI protocols. Journal of Magnetic Resonance Imaging. 2010;31(2):440-6.
32. Kanda T, Ishii K, Kawaguchi H, Kitajima K, Takenaka D. High signal intensity in the dentate nucleus and globus pallidus on unenhanced T1-weighted MR Images: relationship with increasing cumulative dose of a gadolinium-based contrast material. Radiology. 2014;270(3):834-41.
33. Quarles C C G J. Repeatable first pass DSC-MRI measurements using saline as a reverse-effect contrast agent. ISMRM Proceedings. 2009.
34. Deli M, Fritz J, Mateiescu S, et al. Saline as the sole contrast agent for successful MRI-guided epidural injections. Cardiovasc Intervent Radiol. 2013;36(3):748-55.

We claim:

1. A method comprising:
administering an aqueous contrast agent to the vascular system of a subject, and
performing a magnetic resonance scan to detect the MR signal enhancement effects of the aqueous contrast agent, wherein the magnetic resonance scan comprises applying at least one pulse sequence selected from a PD-weighted pulse sequence, a T₁-weighted pulse sequence, a T₂-weighted pulse sequence, and a D-weighted pulse sequence.

2. The method of claim 1, wherein the magnetic resonance scan comprises applying a T₁-weighted pulse sequence.

3. The method of claim 2, wherein the magnetic resonance scan further comprises applying at least one pulse sequence selected from a PD-weighted pulse sequence, a T₂-weighted pulse sequence, and a D-weighted pulse sequence.

4. The method of claim 1, further comprising comparing the MR signals at a first period with the MR signals at a second period, wherein the first and second periods are different and are selected from before administration of the aqueous contrast agent, during administration of the aqueous contrast agent, and after administration of the aqueous contrast agent.

5. The method of claim 1, wherein the presence of the aqueous contrast agent in the vascular system of the subject causes at least one of an increase in proton density (PD), an elongation of the longitudinal magnetization recovery time (T₁), an elongation of the transverse magnetization decay time (T₂), and an increase in the diffusion coefficient (D) in the dipolar relaxation signal from the subject.

6. The method of claim 2, wherein the presence of the aqueous contrast agent in the vascular system of the subject causes an elongation of the longitudinal magnetization recovery time (T₁).

7. The method of claim 1, wherein the aqueous contrast agent enhanced dipolar relaxation signal from the subject is enhanced by at least about 20%.

8. The method of claim 1, wherein the aqueous contrast agent enhanced dipolar relaxation signal from the subject is enhanced by at least about 30%.

9. The method of claim 1, wherein the aqueous contrast agent is saline solution.

10. The method of claim 1, wherein the aqueous contrast agent is distilled water.

11. The method of claim 1, wherein the aqueous contrast agent does not comprise blood protein.

12. The method of claim 1, wherein the aqueous contrast agent does not comprise albumin.

13. The method of claim 1, further comprising generating a contrast-enhanced magnetic resonance image of the subject.

14. The method of claim 13, wherein the contrast-enhanced magnetic resonance image of the subject is generated by a method comprising forming a map of at least one of maximum enhancement (maxENH), area under the curve (AUC), time to peak (TTP), and mean transit time (MTT).

15. The method of claim 1, wherein the magnetic resonance scan is a quantitative magnetic resonance scan.

16. The method of claim 1, wherein the magnetic resonance scan is a functional magnetic resonance scan.

17. The method of claim 1, wherein the magnetic resonance scan is a dynamic magnetic resonance scan.

18. The method of claim 1, wherein the magnetic resonance scan is a magnetic resonance angiographic (MRA) scan.

19. The method of claim 1, wherein the method comprises performing at least one of perfusion imaging, arterial spin labeling, and diffusion imaging.

20. The method of claim 1, wherein the method comprises performing a magnetic resonance angiogram or venogram of the subject.

21. The method of claim 1, wherein the subject is a member of at least one group in which use of a gadolinium containing contrast agent is contraindicated.

22. The method of claim 1, wherein the signal enhancement is not dependent on the presence of elevated deuterium in the contrast agent.

23. The method of any one of claims 1-22, further comprising recording the MR signal enhancement effects of the aqueous contrast agent.

24. A system for performing perfusion MRI, comprising an aqueous contrast solution and an injection apparatus configured to provide a maximum injection rate of the aqueous contrast solution to a subject vascular system of at least about 5 ml/s.

25. A system for performing perfusion MRI, comprising an aqueous contrast solution, an injection apparatus, and a controller comprising a programming system to allow programming of an injection protocol that specifies an injection rate of the aqueous contrast solution to a vascular system of a subject at a maximum rate of at least about 5 ml/s.

26. The system of claim 25, wherein the programming system comprises a computer.

27. The system of claim 26, further comprising computer usable media having a computer readable program code embodied therein, said computer readable program code specifying injection of a bolus of the aqueous contrast agent into the vascular system of a subject at a maximum rate of at least about 5 ml/s.

28. The system of claim 25, further comprising a processor configured to generate a contrast-enhanced magnetic resonance image of the subject by a method comprising generating a map of at least one of maximum enhancement (maxENH), area under the curve (AUC), time to peak (TTP), and mean transit time (MTT).
